(19) United States
(12) Patent Application Publication
(10) Pub. No.: US 2023/0109873 A1
(43) Pub. Date: Apr. 13, 2023

(54) DEVICES, METHODS, AND SYSTEMS FOR ELECTROPORATION USING CONTROLLED PARAMETERS

(71) Applicant: CyteQuest, Inc., Ithaca, NY (US)

(72) Inventors: Thomas N. Corso, Groton, NY (US); Harold G. Craighead, Ithaca, NY (US); Jacob Vanderburgh, Ithaca, NY (US)

(21) Appl. No.: 17/965,255

(22) Filed: Oct. 13, 2022

Related U.S. Application Data
(60) Provisional application No. 63/255,287, filed on Oct. 13, 2021.

(51) Int. Cl. C12M 1/42; B01L 3/00; C12N 15/87

(52) U.S. Cl. CPC C12M 35/02; B01L 3/502761; C12N 15/87; B01L 2200/0652; B01L 2300/0816; B01L 2300/0645; B01L 2300/0864; B01L 2300/18; B01L 2300/0663; B01L 2400/082; B01L 2400/0475; B01L 2300/0829; B01L 2400/0424

(57) ABSTRACT

Disclosed are microfluidic flow-based electroporation systems that have a flow device, an electrical control module, a fluid delivery module, and a multi-well module. The systems can be used in methods of selecting an electroporation parameter, and in methods of electroporating cells using the selected parameters.

### CROSS-REFERENCE TO RELATED APPLICATIONS

This application claims the benefit of priority to U.S. Provisional Application No. 63/255,287, filed Oct. 13, 2021; the entire contents of said application are incorporated herein by reference.

### BACKGROUND

Electroporation is a process used to modify cells by insertion of biomolecules. These cell modifications are important for biological and biomedical research. These cell modifications also form the basis of a new class of cell-based therapies that require careful and reliable treatment of cells.

There exist various systems for electroporation. As an example, there are those that use cylindrical pipette tips with electrodes on the inner surface of the pipette. However, these cylindrical devices lack the scalability, flow control, and uniformity of electric field to which the cells are exposed for electroporation.

Because of the importance of cell modifications enabled by electroporation, there is a need in the field for improvements to controlling and optimizing various parameters used during electroporation.

### SUMMARY

This invention relates, in some aspects, to a new approach to electroporate cells using fluid flow in a microfluidic device. The microfluidic devices and systems can be configured to realize various improvements in the electroporation process related to the ability to efficiently handle small samples and to efficiently handle multiple samples of different types either simultaneously or in parallel. Process conditions developed for the small samples in flowing systems may readily be translated to larger volume systems for modifying large numbers of cells using the processes optimized efficiently with small samples, which is a unique scaling capability of the disclosed approach. We describe here several conditions and approaches used to realize these advantages, which include controlling fluids and applied voltages for electroporation. The disclosed systems, devices, and methods create substantial advantages in the ability to modify cells by insertion of bio-active molecules.

In some aspects, microfluidic flow-based electroporation systems include a flow device that includes a planar flow channel, a pair of electrodes, a first port, and a second port; an electrical control module that connects to the pair of electrodes; a fluid delivery module that links to the flow device at the first port; and a multi-well module that links to the flow device at the second port.

In some embodiments, the multi-well module includes a 6-, 24-, 48-, 96-, or 384-well plate. In some embodiments, the fluid delivery module allows moving fluid from the first port toward the second port, from the second port toward the first port, or from either port toward the other port. In some embodiments, the fluid delivery module includes a syringe. In some embodiments, the fluid delivery module links to the flow device at the first port via a first tubing. In some embodiments, the multi-well module links to the flow device at the second port via a second tubing. In some embodiments, these systems further include a robotic module that moves the multi-well module relative to the flow device. In some embodiments, these systems further include a robotic module that moves the flow device relative to the multi-well module.

In some embodiments, the fluid delivery module includes a plurality of fluid inputs. In some embodiments, the plurality of fluid inputs includes at least one with cells, at least one with biological molecules, and at least one with a buffer composition. In some embodiments, the biological molecules include nucleic acids. In some embodiments, the flow device includes an integrated fluidic output nozzle that links to the multi-well module. In some embodiments, the flow device includes a plurality of planar flow channels. In some embodiments, the flow device includes a single planar flow channel.

In some aspects, methods for optimizing an electroporation parameter include running cells and biological molecules through any of the disclosed systems; determining transfection efficiency, viability, or both for the cells; and selecting an electroporation parameter based on the determined transfection efficiency, viability, or both.

In some embodiments, the electroporation parameter includes cell type, cell concentration, biological molecule type, biological molecule concentration, fluid flow rate, fluid volume, buffer type, voltage amount, voltage waveform, collection time, or a combination thereof. In some embodiments, voltage waveform, collection time, or a combination thereof is varied during a run of the cells and the biological molecules through the system. In some embodiments, cell type, cell concentration, biological molecule type, biological molecule concentration, fluid flow rate, fluid volume, buffer type, or a combination thereof is varied across different runs of the cells and the biological molecules through the system but kept constant during a run of the cells and the biological molecules through the system.

In some embodiments, the methods include moving the multi-well module between runs or during a run of the cells and the biological molecules through the system to collect different samples in different wells. In some embodiments, the methods include running a plurality of types of cells, biological molecules, or both through the system using a plurality of fluid inputs. In some embodiments, the biological molecules include nucleic acids. In some embodiments, the running is toward the multi-well module. In some embodiments, the running is away from the multi-well module.

In some aspects, methods for electroporating cells include running cells and biological molecules through any of the disclosed systems using a parameter determined via any one of the methods for optimizing an electroporation parameter, and collecting transfected cells.

In some embodiments, the electroporation parameter includes cell type, cell concentration, biological molecule type, biological molecule concentration, fluid flow rate, fluid volume, buffer type, voltage amount, voltage waveform, collection time, or a combination thereof. In some embodiments, the running is toward the multi-well module. In some embodiments, the biological molecules include nucleic acids.

### BRIEF DESCRIPTION OF THE DRAWINGS

FIG. 1 shows a block diagram of a typical system to test various electroporation conditions. The Flow chip (i.e., flow device) is the microfluidic electroporation chip with a flow channel and electrode pair. The blue lines represent tubing with the arrows indicating the direction of flow. In this case multiple samples can be collected that have been treated by application of possibly different voltage waveform.

FIG. 2A-FIG. 2C show the results from a voltage waveform test. (FIG. 2A) Representative waveform used to electroporate Jurkat cells. (FIG. 2B) Flow cytometry graphs depicting measurement of viability (7-AAD) and transfection efficiency (GFP). Representative graphs are shown for control and electroporated cells. (FIG. 2C) Plot depicting GFP expression and viability from Jurkat cells electroporated with 7V pulses (depicted in panel A) of varying pulse durations. The disclosed platform achieves 97% GFP expression with 95.2% viability with the 5V, 1500 us waveform.

FIG. 3 shows the system configuration, and varying fluid composition in addition to electrical parameters. The system has multiple independently controlled fluid sources. By varying the flow rates from the different fluid sources, different fluid compositions can be formed by combination near the electroporation chip input. The multiple flows, in one embodiment of the chip can form laminar layers of flow with different compositions in each layer.

FIG. 4A-FIG. 4B show a sketch of a microfluidic electroporation chip with an integrated fluidic output structure or nozzle. The dashed lines indicate the fluid channel in the chip. FIG. 4A shows the nozzle on the planar face of the chip. FIG. 4B shows the nozzle on the end. In both cases the output could be dripped from the output or forced under pressure into another fluidic element.

FIG. 5 shows a sketch of a microfluidic electroporation chip with an integrated fluidic output structure or nozzle. In this case the processed sample is directed into a well on a microfluidic plate. Other sample collection volumes are possible.

FIG. 6 shows a diagram of multi-channel system that allows processing of multiple independent samples simultaneously. The pumps can move fluid in either direction.

FIG. 7A-FIG. 7C show exemplary multiplexed electrical parameters and collecting the electroporated cells in a multi-well plate.

FIG. 7A shows multiplexing electrical parameters by automated, computer controlled, variation of waveform parameters and depositing the electroporated cells by a robotic control in different wells of a 24-well plate. This facilitates rapid analysis of effects of different electrical parameters of electroporation. The arrow shows the order of dispensing cells from the flow device into the plate.

FIG. 7B shows multiplexing electrical and chemical parameters and illustrates how more than one chip can be operated simultaneously (here two chips) to test different liquid compositions and vary electrical parameters for optimization. FIG. 7B shows 2-channel capability. The system has a dual syringe pump for multiplexing electrical parameters. The system is capable of automated, computer controlled, variation waveform parameters, and depositing cells by a robotic control in different wells of a 24-well (or 96 well) plate. This facilitates rapid analysis of the effects of different electrical parameters of electroporation using two different chips. This system has one dual syringe pump, one robot, and a two-channel waveform generator. The arrow shows the order of dispensing cells from the flow device into the plate. Two arrows indicate the use of two flow devices that dispenses cells independently.

FIG. 7C shows a variation of the system with an 8-channel pump to simultaneously test 8 different liquid conditions while varying electrical parameters. With multi-channel D/A converters, multiple independent electrical and chemical parameters can be tested simultaneously. This system allows multiplexing electrical parameters, and is capable of automated, computer controlled, variation waveform parameters, and depositing by robotic control in different wells of 96-well (or other multi-well) plate. This facilitates rapid analysis of the effects of different electrical parameters and chemical parameters independently. The arrow shows the order of dispensing cells from the flow device into the plate. 8 arrows indicate the use of 8 flow devices that dispenses cells independently. This system may comprise an 8-syringe pump, a robotic module, and one or more voltage channels.

FIG. 8A-FIG. 8C shows electroporation platform for rapid optimization of time varying electrical waveforms. (FIG. 8A) Schematic of the electroporation flow cell depicting the sandwich assembly. (FIG. 8B) Photograph of an assembled electroporation flow cell (FIG. 8C) Entire platform with pictured syringe pump, 0.02″ inlet and outlet tubing, robot capable of automated translation in three axes, flow cell with electrical attachments, and 96-well plate.

FIG. 8D shows a side view of an exemplary flow device.

FIG. 8E shows a plot of the bipolar square wave used as the time varying waveform for this optimization.

FIG. 8F shows sample electroporation flow cytometry plots from electroporated Jurkat cells expressing green fluorescent protein. (panel A) Cell count measured by excluding debris through forward and side scattering signal via flow cytometry (panel B) Cell viability measured by incubation with the 7-AAD viability dye and measurement of positive staining cells via flow cytometry (panel C) Transfection efficiency measured by live-gated GFP positive cells.

FIG. 9A-FIG. 9B show an exemplary system. FIG. 9A shows multiplexed optimization of transfection parameters using up to 8 electroporation flow cells and 96-well plates. The system comprises an 8-channel pump, a computer and integrated software for 8 digital V(t) outputs, a pump control, a robot scan control, a 8-channel digital-to-analog converter (DAC), a 96-well plate, 8-channel Amp, and 8 independent voltage(t) outputs. The 96-well plate is scanned by a robotic module in a computer-controlled manner. FIG. 9B show the same system as FIG. 9A but shows a schematic diagram of the system using 2 flow devices. It exemplifies the versatility of the system—any desired number of the flow devices can be used.

FIG. 10 shows a 96-well-plate-pitch compatible flow device that can be used with a robotic module. The flow device in this figure uses low volume of fluid for efficient multiplexing. Here, the port on the bottom of the flow device can be used for dispensing the electroporated cells as shown in FIG. 8B. Alternatively, the same port can be used to collect fluid or cells from a multi-well plate. This symmetric design of the flow device allows the user to carry out electroporation with flow in either direction.

FIG. 11 shows a schematic of cell processing function that is integrated with a planar fluidic electroporation device (which, for example, can be a microfluidic chip or the like). The fluidic flow device schematic shows separate regions for magnetic cell selection and electroporation in a planar format.

FIG. 12 shows a system of an independent microfluidic device and a planar electroporation device (e.g., an electroporation chip). In this figure, and in the rest of the disclosure, unless described otherwise in a particular context, the word “chip” is used interchangeably with the word “device” for facilitating the discussion of various aspects.

FIG. 13 shows an automated cell manufacturing platform that incorporates an electroporation system.

FIG. 14 shows a multi-flow device with dynamic control of processes to obtain optimized cell modification parameters.

FIG. 15 shows a schematic of independently controlling and varying the chemical composition of three fluid streams for combinatorial processing.

### DETAILED DESCRIPTION

Multi-well plates, such as 6, 24, 48, 96, 384 well plates, are frequently used in biotech research and industrial processes for their efficiency in parallel processing of multiple samples. The plates are standard formats used in automated sample handling. We have demonstrated the ability to interface the disclosed flow-based electroporation systems for dispensing electroporated samples that have been treated using different conditions during a single run. This has the advantage of enabling the user to rapidly test conditions for optimal results, usually described by the transfection efficiency of modified cells and the viability or health of the resulting modified cells.

The disclosed systems can be used with multi-well plates as the source of cell solutions and reagents for the electroporation process. This has the utility of interfacing to standard sample processing formats and enhances the ability to rapidly handle large numbers of small samples.

Various aspects of these systems and methods are illustrated in the provided examples. The flow devices described here can have many configurations. The simplest contains a single uniform flow channel and a single pair of electrodes. Also encompassed are devices with multiple channels and a single pair of electrodes connecting to each channel. Multiple electrodes can readily be incorporated in the channels with independently addressable connections. The channels can also be made in different configurations with additional functions such as varying the width or thickness of the flow channel(s) to apply hydrodynamic forces to cells in addition to or instead of electric fields. Integrating with other on-chip microfluidic device functions such as cell sorting or filtering is also possible.

### Definitions

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

All numerical ranges provided herein are understood to be shorthand for all of the decimal and fractional values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9 and all intervening fractional values between the aforementioned integers such as, for example, ½, ⅓, ¼, ⅕, ⅙, ⅛, and 1/9, and all multiples of the aforementioned values. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “chip” is used interchangeably with the term “device” or “flow device.”

As used herein, the term “voltage waveform” (and related terms, e.g., “time-dependent voltage waveform,” “temporal voltage wave form”) refers to the voltage that varies in time as supplied to the electrodes by the voltage controller or other source of voltage. This may be described by a periodically repeated time varying function, but it can also vary arbitrarily in time and not be repeated.

### Methods

The invention relates, in some aspects, to methods of selecting one or more electroporation parameters. These methods include running cells and biological molecules through any of the encompassed systems, then determining transfection efficiency, viability, or both for the cells, and then selecting one or more electroporation parameters based on the determined transfection efficiency, viability, or both.

In various embodiments, a user may select the parameters according to a preferred objective function (e.g., maximum transfection efficiency only, maximum cell viability only, maximum transfection efficiency and viability combination, maximum transfection efficiency and viability combination with each weighed non-equally). In some embodiments, a user may also consider other factors, such as the cost of reagents or conditions used, when selecting the parameters.

In some embodiments, the one or more electroporation parameters include cell type, cell concentration, biological molecule type, biological molecule concentration, fluid flow rate, fluid volume, buffer type, voltage amount, voltage waveform, collection time, or a combination thereof. Among these, in some embodiments, voltage waveform, collection time, or both are varied during a run of the cells and the biological molecules through the system. This allows a more efficient scanning of the parameter space as compared to keeping these parameters constant during a single run. Other parameters, such as cell type, cell concentration, biological molecule type, biological molecule concentration, fluid flow rate, fluid volume, buffer type, or combinations of these can be kept constant during a run of the cells and the biological molecules through the system. However, these other parameters can also be varied across different runs of the cells and the biological molecules through the system to allow additional scanning of the parameter space.

The described systems enable efficient scanning of the parameter space using these methods, for example by allowing collection of different samples in different wells of the multi-well module and by running different types of cells or biological molecules from multiple fluid inputs. In these methods, the multi-well module can be used to collect the output samples as well as to contain the input samples, since the fluid delivery module can move the fluid in either direction.

Once preferred parameters are known (e.g., selected using the described methods), cells can be electroporated by running them and biological molecules through the described systems using the selected parameters and then collecting the transfected cells.

In some embodiments, the biological molecules are nucleic acids, such as DNA or RNA (e.g., mRNA), but other types of molecules, such as small molecules or polypeptides can also be introduced into the cells using the described methods.

### Flow Device for Electroporation

In some embodiments, an electroporation device comprises at least one planar flow channel flanked by at least one pair of electrodes on opposite sides of the channel to which electrical potentials can be applied to create an electric field across the channel between the electrode pair. In some embodiments, the dimensions of the fluid channel combined with the characteristics of the fluid flow provide sufficient control to maintain individual living cells within the fluid flow at similar positions with respect to proximity to the electrode pair they are passing through. As the living cells flow through the channel between the electrodes, the distance from the cell to each electrode is held to be nearly constant and in a manner that prevents one living cell from shielding another living cell from the applied electric field. In some embodiments, the cell flow is one layer thick in the channel dimension between the opposing electrode pairs so that the cells are independently exposed to the same electrical current formed in the channel when passing between the electrode pairs. The channel, in some embodiments, has no restriction on distance in the other two dimensions of channel length and opposing channel walls not flanked by the electrodes. The cells flow through the channel at a set flux, and these features enable a user to apply precise electric fields to the cell. The strength of the electric field is strong enough to form pores within the membrane of the living cell through which heterologous objects (e.g., biological molecules) can traverse the cell membrane, but weak enough to not lyse the cell.

The device includes one or more ports (e.g., fluid inputs and fluid outputs). When the device includes a single fluid input, a single laminar fluid stream is created. The single fluid stream contains living cells in combination with a heterologous object (e.g., biologically active molecules) for introduction of the heterologous object (e.g., biologically active molecule) into the living cell by electroporation. Suitable spacing between the electrodes includes about 2 to 5 times larger than the diameter of the cell, or smaller than approximately two times the typical cell diameter, forcing the cells to pass through the space between the electrodes in a single layer. The living cells in the single fluid flow are maintained in a similar position as other living cells as they pass through the electric field between the electrode pairs, so each cell is exposed to similar electrical and chemical conditions during electroporation. Suitable distance between the electrodes of an electrode pair includes a range of from about 50 micrometers to about 100 micrometers, or less than about 100 micrometers (e.g., 20, 25, 30, 35, 40, 45, 50, 55, 60, 65, 70, 75, 80, 85, 90, 95 micrometers).

When the device includes at least two fluid inputs, multiple laminar sheath fluid streams are created. Each fluid input can accept a separate fluid stream. For example, one stream contains living cells, and another contains the biologically active molecules. Thus, the living cells and the biologically active molecules flow through separate fluid inputs into the channel. The streams are separated by laminar sheath flow. The dimensions of the fluid channel are constructed to accommodate the laminar flow separated streams so that living cells contained in the fluid flow are maintained in a similar position as other living cells as they pass through the electric field between the electrode pairs. In a system with multiple sheath flows, the sheath flows separate the cells from the electrode and channel walls with a constant spacing controlled by the flow rates. The multiple sheath flow devices allow the chemical composition of the fluid on opposite sides of the cell to differ permitting an efficient electrical drive of charged molecules such as DNA and RNA into the cells. The flow of liquid through the channels can be unvarying in time, which simplifies the process and assures that all cells experience the same combination of conditions during electroporation as they pass through the flow channel. Suitable distance between the boundaries of the sheath flow containing the living cells between paired electrodes includes from about 50 micrometers to about 100 micrometers; less than about 100 micrometers (e.g., 20, 25, 30, 35, 40, 45, 50, 55, 60, 65, 70, 75, 80, 85, 90, 95 micrometers); about 2 to 5 times larger than the diameter of the cell; or smaller than approximately two times the typical cell diameter, forcing the cells to pass through the space between the electrodes in a single layer. The device can contain one or more fluid outputs. In some such embodiments, the distance between the electrode pairs can be greater than that noted above since the living cells in the sheath stream are maintained in a similar position as other living cells as they pass through the electric field between the electrode pairs by the adjacent sheath flows. A suitable distance between the electrodes of an electrode pair includes from about 50 micrometers to about 500 micrometers. Another advantage of an embodiment of the disclosure is that the user can manipulate the chemical and electrical properties of the environment at different positions along the length of the channel. Furthermore, some embodiments of the disclosure allow the user to monitor various properties of the cells and/or the solution to modify and optimize the flow and voltage parameters in real time.

In some embodiments, a flow device for electroporation comprises at least 1, 2, 3, 4, 5, 6, 7, 8, 9, 10, 11, 12, 13, 14, 15, 16, 17, 18, 19, or 20 planar flow channels. In some embodiments, a flow device comprises 1 planar flow channel.

In some embodiments, a flow device for electroporation comprises at least 1, 2, 3, 4, 5, 6, 7, 8, 9, 10, 11, 12, 13, 14, 15, 16, 17, 18, 19, or 20 pairs of electrodes. In some embodiments, electrical current is applied to only one pair of electrodes.

In some embodiments, a flow device comprises at least one pair of electrodes that extend to at least one edge of the flow device such that electrical control module can connect to the electrodes at one end of the planar device. In some embodiments, the at least one pair of electrodes extend to the edge of the flow device that is distal to the at least one second port. In some such embodiments, the at least one second port may be the outlet port that dispenses electroporated cells into a multi-well module.

In some embodiments, a flow device comprises at least one first port and at least one second port. In some embodiments, the fluid flows into the at least one first port and flows out of the at least one second port. In other embodiments, the fluid flows into the at least one second port and flows out of the at least one first port. Accordingly, the flow may enter into and flow out of either of the first or second port. In some embodiments, a flow device comprises at least 1, 2, 3, 4, 5, 6, 7, 8, 9, 10, 11, 12, 13, 14, 15, 16, 17, 18, 19, or 20 first ports, thereby facilitating flowing in or out of multiple streams of fluid. Similarly, in some embodiments, a flow device comprises at least 1, 2, 3, 4, 5, 6, 7, 8, 9, 10, 11, 12, 13, 14, 15, 16, 17, 18, 19, or 20 second ports.

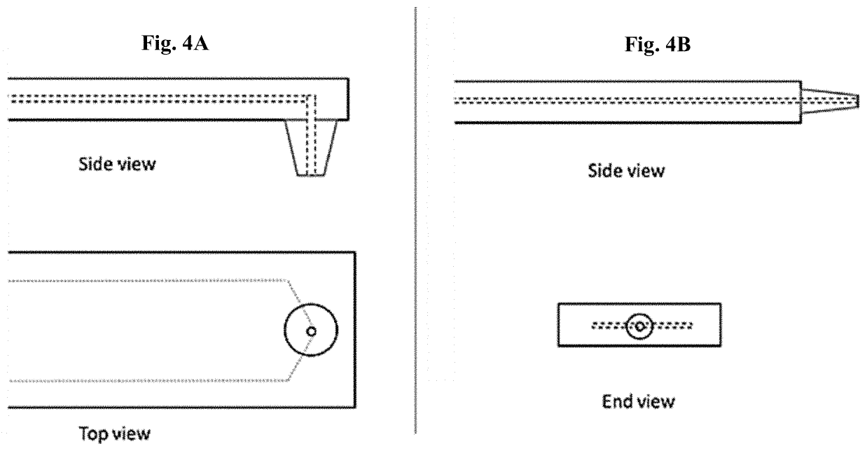

In some embodiments, a flow device comprises at least one port that comprises the shape and location of the port as depicted in FIG. 4A or FIG. 4B.

For example, in some embodiments, the at least one second port is located: (a) on the bottom of the flow device (e.g., FIG. 4A) such that the flow from the second port is perpendicular to the flow within the channel; or (b) on the side of the flow device (e.g., FIG. 4B) such that the flow from the second port is in the same direction as the flow within the channel.

In some such embodiments, the at least one first port is located on the bottom of the flow device, on top of the flow device, on the side of the flow device, or any combination thereof.

In some embodiments, the width of the device is at least about 1, 2, 3, 4, 5, 6, 7, or 8 mm but no more than about 9, 10, 11, 12, 13, 14, 15, 16, 17, 18, 19, or 20 millimeters (mm). In preferred embodiments, the width of the device is about 8 mm. In other preferred embodiments, the width of the device is about 6 or 7 mm.

### Channel

By limiting the gap dimension between electrodes to be about 2 to 5 times larger than the diameter of the cell or less than approximately two times the cell diameter, there is not enough physical space for more than one cell in the flowing stream to be located in the channel gap between the electrodes. This controlled gap spacing as well as operating in the laminar flow regime (e.g., no turbulent flow) allows for controlled positioning of a single given cell between the electrodes in this one plane. While the flow channel is narrow in proximity to the electrodes, the channel can be made as wide as necessary in the orthogonal dimension to achieve the desired flow rate of the cells through the channel. Similarly, the length of the channel, in some embodiments, has no restrictions. Control of the distance between the electrodes allows each cell to be isolated or held in a similar position relative to the electrodes. Thus, each cell is essentially subjected to a similar electrical and chemical environment while, at the same time, high total cell throughput is possible. In some embodiments of the device, the channels can be manufactured so that distance between the support blocks, and thus the electrodes, can be adjusted to accommodate different types and sizes of living cells. The support blocks on which the electrodes are mounted can be made of any nonconductive or electrically insulating material, such as glass, plastic, or optically transparent material.

In some embodiments, the channel width is at least about at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, or 7 mm. In some embodiments, the channel width is no more than about 8 mm, 7 mm, or 6 mm. In some embodiments, the channel width is no more than about 6 mm. In some embodiments, the channel width is at least about 2 mm but no more than about 7 mm. In some embodiments, the channel width is about 2 mm.

In some embodiments, the channel height is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 millimeter (mm). The height, in some embodiments, is about 40, 50, 60, 70, 80, 90, 100, 110, 120, 130, 140, 150, 160, 170, 180, 190, or 200 micrometers (μm). In some embodiments, the height is 80 micrometers. In some embodiments, the height is 100 micrometers.

### Systems

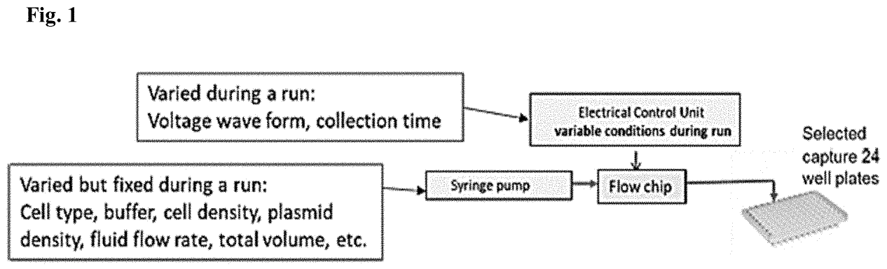

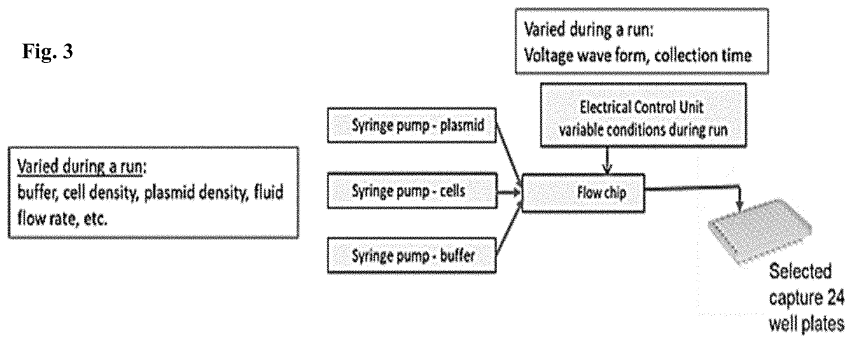

The invention relates, in some aspects, to microfluidic flow-based electroporation systems. The system comprises at least one flow device of the present disclosure, which may alternatively be referred to as a flow chip, for example as in FIG. 1 and FIG. 3. As described above, in some embodiments, a flow device comprises at least one planar flow channel, at least one pair of electrodes, at least one first port, and at least one second port. In some embodiments, the flow device comprises two or more planar flow channels.

The system may also comprise at least one electrical control module (which supplies and/or controls voltage applied to the electrodes) that connects to at least one pair of electrodes.

U.S. Pat. Appl. Publ′n No. 2020/0399578 is incorporated herein by reference.

The system may additionally comprise at least one fluid delivery system that connects to the flow device at the first port. The fluid delivery system can be a syringe, or a more complicated device for moving the fluid in a direction—toward the first port only, toward the second port only, or toward either port as chosen by a user. The fluid delivery system can be connected to the flow device via a tubing. In some embodiments, the fluid delivery module supplies two or more fluid inputs to the flow device. Having two or more fluid inputs can be useful, for example to supply cells, biological molecules (e.g., DNA, RNA), buffer through separate fluid inputs.

The system may also comprise a multi-well module that connects to the flow device at the second port. The multi-well module can be a 6-, 24-, 48-, 96-, or 384-well plate (e.g., standard commercially available plates). The multi-well module can be directly linked to the flow device (e.g., via an integrated fluidic output nozzle of the flow device, see e.g., FIG. 4A and FIG. 4B) or can be indirectly linked (e.g., via a tubing).

In some embodiments, the system may also comprise a robotic module, for example to move either the multi-well module or the flow device relative to each other. This can be used to collect different outputs from the flow device in different wells, or to inject different inputs into the flow device.

Accordingly, in some embodiments, a microfluidic flow-based electroporation system comprises at least one flow device of the present disclosure; at least one electrical control module that connects to the pair of electrodes; at least one fluid delivery system that links to the flow device at the at least one first port; and a multi-well module that links to the flow device at the at least one second port.

In some embodiments, the system comprises at least 1, 2, 3, 4, 5, 6, 7, 8, 9, 10, 11, 12, 13, 14, 15, or 16 flow devices. In some embodiments, the system comprises 8 flow devices. In some embodiments, the flow device is positioned horizontally or vertically with respect to the multi-well module. In some embodiments, the at least one pair of electrodes is connected to the at least one electrical control module from the top, bottom, or the side of the flow device.

### Robotic Module

The system for collecting or dispensing samples from/to a multiwell plate requires a device for moving the output from the electroporation flow cell relative to the wells in a step-wise controlled fashion to access selected wells in the multiwell plate. This “robotic” motion control can be obtained by the use of 1, 2 or 3 motor drives for motion in 1, 2 or 3 dimensions. The motors are actuated by computer control to execute motion in a selected programmed pattern. These motor drives will be coupled to a device for holding the multiwell plate and in the preferred method the multiwell plate is moved beneath the stationary output of the electroporation flow cell. While the robotic motion control can be assembled from individual components, there exists a variety of commercial systems designed for or adaptable to use with multiwell plate. Some examples are listed below.

TABLE 1
Exemplary robotic modules available commercially

| Company | Model No. | Information |
|---|---|---|
| IAI Quality and | TTA-A3G | Tabletop & Laboratory Robots \| IAI |
| Innovation | 3-Axis, Gate type | Automation (iai-automation.com) |
| IAI Quality and | TTA-C3G, | Tabletop & Laboratory Robots \| IAI |
| Innovation | 3-Axis, Cantilever type | Automation (iai-automation.com) |
| igus | XYZ Gantry, Workspace | XYZ Gantry \| Workspace 500 × 500 × |
| | 500*500*100 mm | 100 mm (igus.com) |
| igus | XYZ Gantry, Workspace | XYZ Gantry \| Workspace 400 × 400 × |
| | 400*400*150 mm | 150 mm (igus.com) |
| JANOME | Cartesian robot | Cartesian robot-JR3000ERT-Janome |
| | JR3000ERT | Industrial Equipment-3-axis/cutting/ |
| | | depaneling (directindustry.com) |
| FISNAR | F5200N.2 3-Axis Inline | F5200N.2 Compact Gantry Benchtop |
| | Gantry Robot | Robot-Fisnar |
| | | (fisnar.com) |
| FISNAR | F4200N.2 Compact | F4200N.2 Compact Benchtop Robot- |
| | Benchtop Robot | Fisnar |
| | | (fisnar.com) |
| MACRO | X/Y/Z Cartesian Gantry | X/Y/Z Cartesian Gantry Systems |
| DYNAMICS, | Systems | Archives-Macron Dynamics |
| INC. | | (macrodynamics.com) |
| FISNAR | Cartesian Robot F8800N | Cartesian robot-F8800N series- |
| | series | Fisnar Inc.-4-axis/3-axis/for |
| | | assembly (directindustry.com) |
| IAI Quality and | TTA-A2G | Tabletop & Laboratory Robots \| IAI |
| Innovation | 3-Axis, Gate type | Automation (iai-automation.com) |
| IAI Quality and | TTA-C2G | Tabletop & Laboratory Robots \| IAI |
| Innovation | 2-Axis, Cantilever type | Automation (iai-automation.com) |
| igus | XY Gantry, workspace | XY Gantry \| Workspace 300 × 300 mm |
| | 300*300 mm | (igus.com) |
| MACRO | T-Bot Gantry Robots | T-Bot Gantry Robots \| Macron |
| DYNAMICS, | | Dynamics |
| INC. | | (macrodynamics, com) |
| MACRO | MDS-PSC | MDS-PSC-Macron Dynamics |
| DYNAMICS, | | (macrodynamics, com) |
| INC. | | |
| Epson | RS3 SCARA Robots | Robots For Work \| Epson US |
| Epson | GX-Series SCARA | GX-Series SCARA Robots \| Epson US |
| | Robots | (Epson.com) |
| Epson | T3-B All-in-one SCARA | Robots \| For Work \| Epson US |
| | Robots | (Epson.com) |
| Epson | T6-B All-in-one SCARA | Robots \| For Work \| Epson US |
| | Robots | (Epson.com) |
| Epson | LS-B Series SCARA | Robots \| For Work \| Epson US |
| | Robots | (Epson.com) |
| ABB | IRB 910INV | IRB 910INV \| ABB Robotics- |
| | | Industrial Robots Portfolio \| ABB |
| | | Robotics |
| | | (new.abb.com) |
| ABB | IRB 910SC | IRB 910SC \| ABB Robotics-Industrial |
| | | Robots Portfolio \| ABB Robotics |
| | | (new.abb.com) |
| ABB | IRB 920T | IRB 920 and IRB 920T SCARA robots \| |
| | | ABB Robotics-Industrial Robots |
| | | Portfolio \| ABB Robotics |
| | | (new.abb.com) |
| ABB | IRB 920 | IRB 920 and IRB 920T SCARA robots \| |
| | | ABB Robotics-Industrial Robots |
| | | Portfolio \| ABB Robotics |
| | | (new.abb.com) |
| TPA Motion | Custom OEM XYZ | Plate Robot Four \| Microplate Robot |
| | motion system with a | with Liquid Bath (tpamotion.com) |
| | fourth axis for safety | |
| | during operation | |
| Hudson | Plate Crane Microplate | World Wide Web at |
| Robotics | Handler | hudsonrobotics.com/microplate- |
| | | handling-2/platecrane-ex/ |

### Fluid Inputs and Streams

The fluid can flow through the channel at a rate of 0.1 cm/s, with a relevant range of flow rate between 0.001 cm/s and 10 cm/s. The volume of fluid flowing through the channel relates to the cross-sectional area of the flow channel. For example, for a channel 2 cm wide and 100 micrometers high, the volumetric flow rates would be in the range of from about 0.2 microliters/s to 2 milliliters/s.

The device permits the use of multiple inputs of fluid through slits in the channel device to provide flow with different layers of solution composition. The flow rate of two or more fluid streams into the channel can be controlled to create a sheath flow. Various streams of cells or molecules can enter the channel via these inputs, and these streams can have the same or different flow rates. If desired, streams with different flow rates adopt laminar flow through the channel. Thus, the streams flow in parallel through the channel and remain largely separated, mixing slowly only through diffusion. In this manner, individual cells in the stream of living cells can be isolated between the electrode pair by the laminar flow of the adjacent fluid streams.

The use of multiple inputs of fluid can prevent various types of fouling or contamination. For example, the molecules or nucleic acids to be inserted into the cells can exist in a separate solution from the cells. This can be useful because certain molecules, like RNA, may not be stable in the vicinity of living cells due to enzymes on the cell surface or cell culture media. Also, it is known that degradation of the electrodes can result in the release of contaminants that are toxic to cells. The separate fluid layers ensure that the cells remain free from contaminants from the electrodes. Further, the cells themselves are kept out of contact with both the surface of the support block and the electrodes preventing possible contamination.

In some embodiments, using separate fluid streams allows maintaining different components in media optimal to them for a longer time. For example, one fluid stream can contain the cells to be electroporated, and instead of keeping the cells in a medium that is best for electroporation efficiency, the cells can be kept in a medium that is optimal for them (e.g., for their survival) before electroporation, and then allowed to mix with an electroporation medium during the actual electroporation time window. After the electroporation is complete, the cells can be switched back into the medium that is optimal for them. This allows minimizing the time the cells are in a medium that is not the best for their well-being. The embodiments disclosed herein thus allow dynamically controlling the chemical environment of the cells and the reagents to be electroporated into the cells separately, for example as a function of time and/or position within the fluid channel.

Alternatively, some embodiments of the device can contain a single fluid input through which a homogenous solution of cells and biologically active molecules enter the channel. The stream may consist of a conductive buffer solution containing the biologically active molecules that are to be inserted into the living cells. Compared to the device having multiple inputs, this might be advantageous in that there is a greater opportunity for the cells and the biologically active molecules to come into contact with one another and could increase the efficiency of transformation.

Fluid streams may interface to the device via tubing, fittings, interconnects, a manifold, or discreet fluid path connections. One or more of these parts can be part of the fluid interface. The fluid interface serves to reformat the tubing or conduits into the receiving slit-port of the device. The fluid interface may have changes in surface area as well as varying geometries for delivering fluid to the device. The fluid interface may have features to enhance mixing or maintain laminar flow characteristics. This includes geometric changes that may aid in turbulent flow, diffusion rate changes, or residence time in the flow path. The fluid path may have geometries tailored to avoid the trapping gas (bubbles) or seeding to avoid gas bubble formation due to gas coming out of solution.

The fluid path components may be machined, molded (e.g., injection molding), casted, extruded, or the like. The fluid interface may be fabricated as part of the channel device (one-piece) or bonded (integrated) to the device via a permanent or non-permanent bond. Alternatively, the fluidic interface could be manufactured as part of the device as one integrated component, for example via injection molding where the device and fluid interface are both formed during the molding process. Sealing between the fluid interface and the device may be hermetic, compression-based, O-ring-based, gasket-based, adhesion-based, fused, luer locked (quick connect), flat bottom compression-based, tapered ferrule-based, frusto-conical compression-based, friction fit, barbed connection, or the like.

Fluid transfer lines may be soft, semi-hard, or hard where the leak tight seal between components are made with connections known to those in the art.

Tubing and fluid conduits may be manufactured via extrusion or molding.

For some manifold designs, portions of the system may not contain tubing and fluid will be routed via the manifold structure.

In some embodiments, the fluid interface to the device may be via a leak-tight seal to the planar device with a compressive material such as an O-ring or gasket.

The device can be interfaced to a fluid delivery system. A fluid delivery apparatus or pump is configured to displace fluid from a vessel to establish a fluid flow within the fluid path. The fluid vessel may contain a pure fluid or a solution. The fluid may contain cells, small molecules, or large molecules including chemical entities for the transfection process. The fluid displacement apparatus can provide positive and/or negative displacement of the fluid. This allows fluid to be pushed or pulled through the device and the fluid path components.

The delivery pump may include mechanisms comprising peristaltic, syringe, gear pump, diagram, gas pressure (positive or negative), centrifugal, piston, check-valve, or mechanical displacement, hydrostatic or gravity driven flow.

Preferably, the fluid is indirectly displaced by the pump without the liquid directly contacting any of the moving parts of the apparatus, such as, for example, a peristaltic pump acting upon a fluid filled tube. Alternatively, a positive pressure displacement mechanism may be used where a head pressure displaces liquid from a pressurized vessel, or a negative displacement where a vacuum is used to pull liquid into the electroporation device; vacuum via pressure regulator or a peristaltic pump. The use of negative displacement allows for limited system components to be implemented on the inlet side of the device.

When pulling liquid through the device using negative displacement, an intermediate vessel could be used to capture the cells and fluid exiting the device to avoid contact with the pump's negative displacement equipment (for example, syringe, peristatic pump tube, flow sensor, or the like).

Conversely, fluid may be directly displaced by an apparatus, when the fluid is displaced by directly contacting any of the moving parts of the apparatus, such as, for example, the plunger of a syringe pump. Alternatively, the syringe pump could pull liquid through the device with the target fluids not traveling to the point of reaching the syringe barrel. The syringe may be re-usable or disposable. The syringe may be integrated in the fluid path or connected at the time of use.

Fluid control may be open-loop or may have closed-loop feedback control.

Pumping systems established to date have several weaknesses for controlling flow rate accuracy and precision, and may have performance limitations around controlling stable non-pulsing flows. Controlling of fluid pulsing for the electroporation device is most preferably controlled on the time frame of less than 30 seconds, more preferably less than 10 seconds, and most preferable faster than 1 second. Pulsing control is better than that of 20% for the given time period mentioned in the latter.

For the electroporation device, some preferred embodiments may comprise the peristaltic pump mechanism and or a gas pressure pump-based mechanism. Both types may operate to pull or push liquid.

Traditional peristaltic pumps suffer from high pulsing delivery because the fix rate of mechanical contact on the pump tube via rollers (or linear compression mechanisms) which continuously alter the cross-section area by compressing the tube resulting in tube ID change. Pulsing results from the cross-sectional change of the tube ID. Additionally, peristaltic pumps suffer from accuracy issues that result from tubing compliance changes and tube wear characteristic changes over time and use. This wear is difficult to be compensated or adjusted for without direct measurement of the fluid flow rate or measuring the output with a balance or volumetric measurement. Measuring liquid flow rate with a balance is not preferred, as then an additional instrument must be added that requires an adequate environment (e.g., temperature, humidity, vibration, and space). Also, the fluid path then becomes dependent on access to the relatively large footprint/space requirement of a balance.

Pressure pumps deliver relatively non-pulsatile flow but can suffer from accuracy issues because of fluid path dimensional tolerances, viscosity and temperature changes (fluid and ambient temperature), and liquid height changes as vessels are emptied and filled. Measuring liquid flow rate with a balance is not preferred as then an additional instrument must be added that requires an adequate environment (e.g., temperature, humidity, vibration, and space). Also, the fluid path then becomes dependent on access to the relatively large footprint/space requirement of a balance.

To counter these limitations a flow-sensor may be used to provide closed-loop feedback to the liquid displacement mechanism. Here is proposed the addition of a fluid flow rate sensor (in line with system components) to measure the flow rate in near-real-time with the ability to provide feedback to the fluid displacement mechanism. For example, a flow rate sensor with a peristaltic pump or a gas pressure control system acting on a fluid vessel. The flow rate sensor may control the fluid displacement continuously or intermittently. The sensor may also be used to measure the flow rate as a check in the case of open-loop operation.

Most preferably, in some embodiments, the sensor does not contact the fluid and is not in communication with the device, tubing, or conduit.

The sensor may be reusable where it is used in conjunction with a disposable fluid component(s). Or the sensor may be disposable.

Most preferably, the two types of sensor that may be used include, but are not limited to: (1) ultrasonic-based sensor that is in communication with the fluid path (non-contact), which sensor is in communication with a component the liquid is traveling through; and (2) thermal flow sensor that is in communication with the fluid path (non-contact), which sensor is in communication with a component the liquid is traveling through.

The sensors may be re-used where they temporarily interfaced with a fluid path component that is to be changed, or the sensor may be part of the path and be disposable in nature. In some embodiments, the disposable sensor is integrated in the fluid path.

Interfacing of the liquid entering the device may occur via one or more components, such as a tube or conduit, and/or a fluid interface. The fluid component may comprise one or more features that allow for distributing or altering the flow profile and path of the fluid. This component may a wetted path where the cross-section area and shape may be varying from that of the fluid component exiting cross sectional area or shape. The fluid path change may be part of an assembly or may be molded as part of the electroporation device.

This may include geometric shape(s) that redistribute or format the liquid flow from the tube conduit to a format that is compatible with the device inlet. This architecture of the fluid path depends on the incoming fluid source tubing, fitting, or fluid interface as well as the device fluid inlet shape.

The fluid interface component may be composed of one or more fluid paths and is not limited to the location or number of inlet or outlet features.

A fluidic interface may serve to allow for various formats and types fluid components to make a fluid seal to the microfluidic device inlet. The device inlet may be a circular shape or may have a non-cylindrical geometry or shape. A fluidic interface component may for example allow for one or more incoming fluid lines or conduits to connect to the fluidic interface inlet where the fluid may then traverse a changed cross-section or geometric shape, followed by the fluid exiting the fluidic interface in a cross-section or shape that matches the device fluid inlet geometry. The fluid device inlet geometry would correspond to the fluid interface component output geometry. For example, the fluidic interface may serve to allow for a traditional tube to then supply fluid to a split on the device. Interfacing the fluid can be accomplished in many ways (e.g., different geometric shapes for different types of conduits/tubes), which are available to a person of ordinary skill in the art.

Cells can be manipulated post electroporation. In some embodiments, after electroporation, cells are transferred from the PA to a sterile, multi-well dish or T-flask and allowed to recover for 30-40 minutes at 37° C. The cells are suspended in standard cell medium and either cultured for immediate use or cryopreserved.

In some embodiments, the electroporation device is interfaced to a receiving station that serves the purpose of making one or more connections, providing a leak tight fluidic connection. Such an interface station may also service to make electrical connections.

The receiving station may (1) include ability to make one or more fluidic leak tight connections; (2) include ability to make one or more electrical connections; (3) contain regions allowing for optics or a path for allowing external optics access to the device; (4) have all wetted components that are disposable in nature and compatible with a means of sterilization; (5) have fluid that is isolated via various fluid regions inline valves or more preferably via a non-contact mechanism such as a pinch valve, or the pump may include a way to isolate flow, for example when use of a peristaltic mechanism is utilized (e.g., the wheel may be positioned to pinch a tube or pump tube closed); (6) have conduits, tubing and fluid components that link via barbed or compression fittings; and/or (7) encompass using welding and part melting for manufacturing fluid assemblies.

Cells and the bioactive materials may be presented to the device via several approaches. They may be injected via a robotic fluid handling platform or injection system or connected via biocompatible containers. Bioprocess containers include polymer bags, T-flasks, conical tubes, media bottles, well plates, or the like. These vessels may be one time use or reusable when proper sterilization is performed. Connections to the fluid delivery path may be achieved by compression seals, threaded connections, clamping compression, luer lock mechanisms, O-ring seals, friction seals, gaskets seals, clamping, or similar connections. In the case of pneumatic displacement, the container itself may be pressurized or be contained inside a pressurized vessel.

In some embodiments, the cells may be presented to the device by custom cartridges that interface to the pumping or fluid manipulation system.

### Fluid Output

After electroporation, a mixture of all the fluid streams can leave the device via a fluid outlet. The solution may be transferred to sterile polymer bags, T-flasks, conical tubes, media bottles, well plates, or the like and allowed to recover at 37° C. The cells may then be re-suspended in standard tissue culture medium and plated for immediate use in cellular assays, cryopreserved for future use, or used as desired.

Electrodes with Temporal and Spatial Control of Electric Fields

The separation between electrodes located across the thickness of the fluidic device is small, therefore requiring an applied voltage of only a few volts to perform the electroporation. This contrasts with the need for voltages up to several thousand volts that are normally required for standard electroporation. For example, it is known in the literature that a transmembrane electric field of less than 1 kV/cm is required to porate the cell membrane (Weaver and Chizmadzhev, 1996). For a distance between the electrode pairs of 100 micrometers, this requires approximately a 5 V potential difference to porate an average mammalian cell in accord with the present device. Suitable voltage differences across a living mammalian cell include the following range: 0.1 V to 10 V. For example, for a distance between the electrodes of 100 micrometers this range corresponds to an electric field of 10 V/cm to 1000 V/cm.

The flow channel can have one or several electrically independent electrode pairs. For example, it can have four sets of electrode pairs. Connections to the electrodes are made by using clips or conduction adhesive to connect these to a variable-voltage power supply, function generator, computer via a data acquisition card or amplifier, or batteries with a voltage divider. An ammeter can be used to monitor the current flowing between any pair of electrodes for monitoring and controlling the process.

The electrodes can be configured to apply either a constant, pulsating, or continuously time varying voltage perpendicular to the direction of flow or along the direction of flow. If a pulsating voltage is desired, a pulse duration from about 0.01 millisecond to about 100 milliseconds is suitable. The plurality of electrode pairs can be patterned to create spatially and temporally varying electric fields. The electrodes may be patterned using a photomask in the photolithographic process or by a shadow mask in the sputtering or deposition process. Patterning allows for the fabrication of electrodes with varying geometric shape. The variation of the shape combined with the fluid flow characteristics provides for controlling the time that cells are subject to the electric field.

The invention provides for the ability to pattern electrodes at different locations on the surface of the flow channel that can be individually connected to various electrical sources, where the electrical sources can have different voltage and current characteristics. The disclosed planar fluid systems consisting of electrically insulating material(s) enable the patterning of various electrode structures.

In various embodiments, any one or more of the following can be done: (1) one or more pairs of electrodes can be activated with time-dependent voltage characteristics to open pores in the cells; (2) another pair or more of electrodes can be activated to drive charged molecules into cells; (3) yet another pair or more of electrodes can be used to measure the electrical properties of the cell-containing fluid; (4) another pair or more of electrodes can be used to concentrate nucleic acids or other molecules at the interface between fluid layers of varying conductivity; (5) another pair or more of electrodes can be activated to move the cells actively, or passively by a creating flow in the fluid, to a prescribed location in the flow channel for cell sorting or other purposes; and (6) another pair or more of electrodes can be activated to rotate the cells to increase the surface area exposed for electroporation.

Importantly, disclosed embodiments permit the application of an arbitrary time-varying voltage to different electrodes. The voltage signals can be formed by computer generation of the desired time varying waveform, which is converted to an applied voltage by digital to analog conversion and amplification to the desired voltage range.

A simple waveform could be a sinusoidal voltage of prescribed frequency. The amplitude of the waveform needs to be sufficient to permeabilize the cells. This, in some embodiments, is achieved with a voltage drop of approximately 1 V over the typical 10 micrometers size of a mammalian cell within the fluidic device. The voltage waveform could be about 5V, with a range extending from 0.1 V to 100 V depending on the depth of the fluidic device (e.g., chip) and the ionic composition of the fluid layers. The frequency of the pulse depends on the impedance characteristics of the circuit, specifically on the capacitive aspects of the so-called double layer that is known to form at the surface of the electrode due to the presence of free moving ions in the aqueous solution as well as the resistance of the fluid, or fluid layers of varying conductivity. The impedance of the capacitive double layer depends inversely on the frequency. Consequently, the frequency is preferably around 10 kHz so that the impedance of the fluid layers dominates, leading to most of the voltage change occurring within the fluid layer and not at the electrode-electrolyte interface. The frequency may range from 100 Hz to 1 MHz depending on the fluidic device dimensions and the ionic composition of the fluid layers. The impedance of the circuit may depend on the ionic conductivity of the fluid layers. The resistance of the fluid scales inversely with the ionic concentration, while the double layer capacitance is proportional to the ionic concentration raised to some power. The circuit at the electrolyte-electrode interface is often approximated as a capacitor due to the double layer in parallel with a frequency dependent impedance that is in series with a resistance due to charge transferred across the electrode (referred to as the Randles equivalent circuit model). The ability to control the time variation of the voltage means that the current charging the double layer and the current due to charge transferred across the electrodes may be modulated according to the optimum configuration for electroporating the cells. The voltage waveform may also be composed of the sum of a sinusoidal wave in addition to a constant DC voltage offset, resulting in a net flow of current.

Another periodic waveform, according to some embodiments, has a short duration voltage to open pores followed by a lower voltage of longer duration to move charged molecules into proximity to the cells. The movement of the charged molecule can be due to an electrophoretic force, or due to electrophoresis from a net fluid motion induced by the electrodes, or due to a dielectrophoretic force on the charged molecule or cell.

The continuous repeating nature of the waveform is useful for the continuous flow systems. The applied voltage can vary from positive to negative or remain at zero or another constant voltage for portions of the waveform.

A waveform of arbitrary shape can be created by adding together any number of sinusoidal waveforms each with their own frequency and amplitude, in addition to a constant voltage offset.

The net time-average voltage can be chosen to be positive, negative, or zero, providing the ability to control the net direction of charge flow. This would be of utility for controlling surface electrochemistry on electrodes and for directing charged molecules in a chosen direction.

The waveform may also be chosen to open pores in the cells or cell nucleus, and allow time for diffusion of neutral molecules into the cells before another pore-opening voltage application.

In some embodiments, the spatial arrangement of sets of counterpart electrodes across the surfaces of the fluid channel allows creating an electric field within the fluid channel that varies as a function of time and position without a need for a user to create discrete electrical pulses (e.g., via multiple electrical control module providing a waveform to each set of counterpart electrodes, which can be a sinusoidal waveform for any set, and which can be different between the different sets).

Electrodes can be patterned by a variety of methods, including ink jet printing, silk screening, lithographic patterning, vapor deposition through a shadow mask and other methods for patterning electrical conducting material on a variety of substrates including plastics.

### Manufacturing the Device

A person of ordinary skill in the art would understand that there are many ways of fabricating the device or parts thereof using various materials known in the art. Exemplary methods of fabricating the device are presented herein.

Some embodiments of the device are constructed from a three-layer stack of polymer substrates or plastics. All three layers may be laser cut with a small beam spot, high resolution CO₂ laser. The layers on which the electrodes are fixed may be cut from 1 mm thick acrylic slabs, creating opposite surfaces of the channel. A middle layer defines the distance between the electrode pairs. In some embodiments, the three dimensions of the layers are the same. In preferred embodiments, the central layer that defines the channel height is much thinner than the outer two layers on which the electrodes are deposited and which provide the mechanical stability of the device. Although it is most practical for the layers to be the same in dimensions in the plane that the stream flows, these dimensions can be different from one another. One way to manufacture these layers is to use a laser to cut acrylic pieces similar in dimension to a microscope slide 25×75 mm, add fluid inlet slits or ports and add alignment holes to facilitate assembly. A thin film electrode (50 nm) of a gold (Au) is deposited by physical vapor deposition through a shadow mask on the inside surface of each acrylic piece. The middle layer polymer film with medical adhesive on each side is cut to shape and receives the corresponding alignment holes via the laser cutting process. After laser cutting, the three pieces are placed on a jig containing alignment pins corresponding to the alignment holes in each layer. The sandwich assembly is then compression-bonded in a press. This two-step process of laser cutting and compression assembly is amenable to mass production and allows for a cost-effective consumable to be created. The process can be used to manufacture hundreds of thousands of devices per year. This contrasts with many other types of standard non-electroporation microfluidic devices that typically require expensive capital equipment and a large number of chemical processing steps.

Alignment of the device layers may be conducted by optical positioning or a physical means such as alignment pins or structures. The device layers may have receiving features for use with a jig alignment piece or system. Alternatively, the alignment features may reside in the device layers as so no jig or peripheral alignment system is necessary. These may include pin-like structures or features that snap together.

The flow cell could also be produced by an injection molding process to form one or more of the three layers, where the volume can scale to millions of single-use devices per year, using one injection molding press with a multi-cavity mold.

This disclosure allows for architectures for manufacturing the device that are readily amenable to injection molding. In this device, all the layers may be formed via injection molding. The fluidic channel may be formed in one layer at full depth or, alternatively, the channel may span two or more layers, where the full depth is achieved upon assembly. Injection ports may be created via core pins. Alternatively, the fluid inlets may be added post molding as a secondary operation or structure. The layers may be molded from the planar surface or from the edges. Appropriate and efficient part release from the mold cavity is known in the art.

The molded layers may be assembled together through mechanical connection, adhesion, bonding, welding (including ultrasonic and laser), fusing, melting, or the like. Additionally, there may be another material between the layers for connection and sealing such as, but not limited to, a gasket, O-ring, washer, or the like. Alternatively, sealing can be achieved through press tight or bonding features.

Circular entrance ports can be connected with various fittings to conventional tubing such as that from an automated cell manufacturing platform. Low cost manufacturing methods are desirable because the flow cell and material that comes in contact with cell-containing media should typically be discarded after one use to prevent cross-contamination. There are many ways to injection mold including using one mold or more than one over molding technique. Multiple layers may also be bonded post molding using, but not limited to, such techniques as ultrasonic, laser, thermal heat compression, adhesion, or alike.

In some embodiments, the fluid channel may reside in one layer and the opposing sealing structure is a non-injected molded part such as a film, tape, or planar material containing necessary fluid inlets.

In some embodiments, the device may be created by three-dimensional printing or additive manufacturing processes. Other fabrication techniques include compression molding, casting, and embossing.

In some embodiments, devices are made from glass via lithography and wet or dry etching. Alternatively, the devices may be physically machined via computer numeric control (CNC) or ultrasonic machining.

In other embodiments, the devices can be made from various materials, such as, for example, where at least one layer is glass, where at least one layer is plastic, where one of the layers is optically transparent, or where the channel material is electrically insulating.

### Manufacturing the Electrodes

The formation of patterned electrodes on the flow channel surface can be accomplished with a variety of readily available techniques and materials known in the art. Exemplary methods are presented herein.

One method is to use the process of sputtering for deposition of a metallic conducting layer such as gold, platinum, aluminum, palladium, other metals, or alloys of multiple metals. Gold-palladium is an example of a metallic alloy that can be used to compose the electrodes. The electrodes can be made of an optically transparent material to allow observation of the motion of the living cells in the fluid channel of the device. To generate transparent conducting layers, films of indium-tin oxide (ITO) are frequently used. After metal deposition, these conducting layers can be patterned by masking and etching to remove material where it is not wanted to form the desired patterned electrode shapes. Appropriate masks may be formed from photoresist using common photolithographic exposure processes.

One exemplary method for forming electrodes is to deposit electrically conducing films made of metals or other conducting layers such as ITO. By depositing them through a prepositioned mask, sometimes called a shadow mask, the masks are positioned in proximity to the surface to be coated so that the conducting layer reaches the surface only where previously opened regions have been formed in the mask. In addition, a related technique called “lift-off” can be used, in which a patterned photoresist layer can be used to shape the pattern of deposited conducing material. Another exemplary method for patterning deposited electrodes is ablation by laser or ion etching to remove metal to form the electrode pattern.

The deposition of layers of conducting ink can be performed by brushing or spraying, followed by heating to form patterned conducting films.

These thin film patterning processes are well known to those skilled in the art. In this case, the thickness of the films is desired to be in the range of from 5 nm to 5 micrometers, with a preferred range of from 10 nm to 100 nm.

In some embodiments of the device, electrodes can be formed by inlaying wires or metal bars in grooves formed in the support block instead of affixing the electrodes to the support blocks. In this embodiment, grooves are machined into the support block, for example, a plastic support block, and the electrodes are metal. The wires or bars can be formed of metals such as aluminum, nickel, copper, stainless steel, and may be gold plated. The wires or bars may be glued into the groove or held by a tight compression fitting.

Some embodiments of a system include an electroporation device, fluid delivery system including a pump, temperature control and optical and electrical monitor of the cells to obtain real-time feedback on the cell modification process. Feedback can be obtained by monitoring the electrical current passing between the two electrodes to provide information about living cell modifications, imaging of the living cells to provide information about living cell modifications or monitoring fluorescence of the living cells to provide information about living cell modifications.

Some embodiments include a system for inserting a biologically active molecule into a living cell, which system includes an electroporation device capable of performing a cell modification process including inserting a biologically active molecule into a living cell contained in a fluid flow by flowing fluid including living cells and biologically active molecules through a channel between two electrodes, each electrode disposed on opposite sides of the channel; passing the cells through a space between the two electrodes in a single layer so a living cell in the fluid flow is maintained in a similar position as other living cells in the fluid flow as they pass between the two electrodes; and applying an electric voltage across the two electrodes while the living cell is passing between the two electrodes in a manner that prevents one living cell from shielding another living cell from the applied electric field, in which the strength of the electric field to which the living cell is exposed is sufficient to form pores within the membrane of the living cell through which the biologically active molecule can traverse the cell membrane, but not lyse the living cell; a fluid delivery system including a fluid source and a fluid pump in fluid communication with the electroporation device; an electrical current source in electrical communication with the pair of electrodes; a temperature control in thermal communication with in the fluid flow; and an optical and electrical monitor of the living cell capable of obtaining real-time feedback on the cell modification process.

An advantage to the electroporation device is the ability to optically and electrically monitor the cells to obtain real-time feedback on the cell modification process. In some embodiments of the device, a microfluidic electroporation system comprises an observation microscope. Accordingly, the fluid flow controller or voltage controller can be adjusted as required to optimize process efficiency and cell viability. In some such embodiments, the microscope may be positioned so that it views a reservoir that contains biologically active material. For example, this could be nucleic acids. The fluid from input cell reservoir flows through the channel of the microfluidic electroporation device and across the field of view of the microscope, and into a cell collection reservoir, thus enabling the user make adjustments as necessary to improve the efficiency of transformation. As used herein, the cell collection reservoir refers to any vessels, bags, plates, dishes, or containers that are capable of collecting cells flowing out of the flow device.

Temperature control of the solutions or materials in contact with the fluids may be implemented at any instance(s) in the system, including heating and cooling. This may include static control or temperature cycling.

The device can be interfaced to a fluid delivery system. A fluid delivery system (e.g., a pump) operating with a flow controller is configured to displace, preferably, indirectly displace, the fluid from the input cell reservoir to establish a fluid flow within the fluid path. The fluid displacement apparatus can provide positive and/or negative displacement of the fluid. The delivery pump includes mechanisms based on peristalsis, pneumatics (pressure displacement), hydraulics, pistons, vacuum, centrifugal force, manual or mechanic pressure from a syringe, and the like. Preferably, the fluid is indirectly displaced by the pump without the fluid directly contacting any of the moving parts of the apparatus, such as, for example, a peristaltic pump acting upon a fluid filled tube. Alternatively, a pneumatic displacement mechanism may be used where a head pressure displaces liquid from a pressurized vessel. Conversely, fluid may be directly displaced by an apparatus, when the fluid is displaced by directly contacting any of the moving parts of the apparatus, such as, for example, the plunger of a syringe pump.

In addition to common mechanical pumping mechanisms, such as syringe and peristaltic pumps, the fluid delivery means may include gravity driven or hydrostatic pressure driven liquid flow. Here the fluid containing vessel is positioned at a given height (relatively to the device fluid outlet) to provide the desired flow rate. The fluid height is chosen based on the overall system fluid restriction circuit (cross-sectional area, internal diameters, and lengths of the fluid path). In addition to the device's internal flow path dimensions, external components such as tubing internal diameters may be chosen to obtain a desired restriction for controlling the flowrate.

The liquid containing vessel may accept application of controlled gas head-pressure to aid in the displacement of the liquid from the vessel to the device.

The fluid delivery system (e.g., pump) may include a flow sensor for monitoring the flow rate or the flow sensor may provide closed loop feedback to the pump control system. The closed loop feedback can ensure accuracy and reduce pulsing. The pump displaces fluid contained in flexible tubing to create a fluid stream. The system may operate with an inline flow sensor configured to measure directly the fluid flow rate as the fluid passes the sensor. The system, in some embodiments, includes a feedback control in communication with the fluid displacement apparatus and the inline flow sensor. The inline flow sensor measures the flow and communicates with a feedback control mechanism. Suitable types of flow sensor mechanisms include thermal pulse, ultrasonic wave, acoustic wave, mechanical, and the like. The inline sensor may be mechanical-based, electrical-based, motion-based, or microelectromechanical systems (MEMS)-based. The sensor mechanism may be thermal, ultrasonic or acoustic, electromagnetic, or differential pressure. One example of a sensor suitable for use in accord with the present disclosure is a thermal-type flow sensor where the sensor typically has a substrate that includes a heating element and a proximate heat-receiving element or two. When two sensing elements are used, they are preferably positioned at upstream and downstream sides of the heating element relative to the direction of the fluid (liquid or gas) flow to be measured. When fluid flows along the substrate, it is heated by the heating element at the upstream side, and the heat is then transferred non-symmetrically to the heat-receiving elements on either side of the heating element. Because the level of non-symmetry depends on the rate of fluid flow and that non-symmetry can be sensed electronically, such a flow sensor can be used to determine the rate and the cumulative amount of the fluid flow. This mechanism allows the flow to be measured in either direction. In preferred embodiments, the temperature sensors and the heating element are in thermal contact with the exterior of the fluid transporting tube and as the fluid stream only contacts the internal surfaces of the tube, the fluid media avoids direct contact with the sensor and heating elements. This format type allows highly accurate and highly sensitive flow measurements to be performed.

Integration of Fluidic Cell Processing with the Electroporation Device

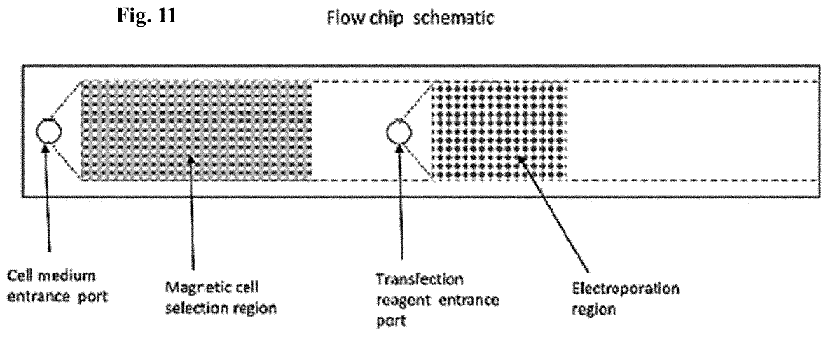

Integration of fluidic cell processing with the electroporation device (e.g., chip) allows building greater function into a system. For example, the multi-channel flow device can incorporate the ability to utilize magnetic bead sorting approaches to select the cells to be processed by electroporation. As an example, FIG. 11 shows a fluidic flow device schematic depicting separate regions for magnetic cell selection and electroporation in a planar format.

Optical transparency of the flow device enables optical monitoring of the processes. Materials may include, but are not limited to, glass, quartz, polymer, metal films on substrate transparent substrates.

Selection of a variety of T-cells and B-cells can be achieved using magnetic beads conjugated with specific antibodies for the given cell type. There are several commercial manufacturers of superparamagnetic beads including Dynal® (polymer beads) and Seradyn of a variety of different sizes, typically between 2 and 5 microns. These beads can be used for the positive selection or depletion from flow of CD8+, CD3+, CD4+, and CD19+ cells, for example. The force (F) on a magnetic particle inside a magnetic field depends on the volume (V) of the particle, the difference in magnetic susceptibility between the particle and the surrounding fluid (Δχ), and on the absolute strength and the gradient of the magnetic field (B): F=V·Δχ(B·∇)B. By establishing a large magnetic field and a large magnetic field gradient within the fluidic device, cells that have bound magnetic beads conjugated with the appropriate antibody can be held stationary relative to the flow. In this case, the magnetic force must be stronger than the drag force on the bead from the flow and able to overcome the bead's random diffusive motion.

To establish the sufficient magnetic field within the fluidic device, small neodymium iron boron (NdFeB) magnets featuring magnetic flux densities of up to 500 mT at the pole surface can be placed in proximity to the planar surface. Commercially available versions of these magnets allow for the manipulation of magnetic particles or cells inside a microchannel even when the magnet is placed at several mm distance from the channel. Removing the magnet from proximity of the surface will release the cells. These magnets can be purchased in a variety of sizes ranging from 0.01 to 10 cm in diameter and different geometrical shapes including cylinders, cubes, rings, etc. Also, commercial electromagnets may be used to establish the necessary field and gradient though Joule heating from the relatively high current make these more problematic in small volume applications. It is also possible to fabricate an electrode on the planar surface by the deposition of a conducting metal like gold or platinum. Additionally, the magnetic field within the fluid can be enhanced in the presence of an external magnetic field by depositing and patterning magnetic metals (typically nickel or iron) within the fluid layer. Removing the magnet from proximity of the surface will release the cells. Alternatively, the device and/or the magnetic source may be movable.

Measuring properties of cells upstream and downstream to measure the effectiveness of the electroporation. This requires flow and the ability to constantly monitor the electrical properties (e.g., resistance) of the cell-containing liquid. One could also monitor the change of resistance during the electroporating pulse, where the time-dependent I-V relationship would provide information on the effectiveness of the electroporation process. This is unlike other systems that may exist that do not monitor the effects during the continuous flowing process.

Post Electroporation Cell Manipulation: After electroporation, cells may be moved to an additional region in the device for secondary processing or transferred. The cells may be transferred from the device fluid outlet (or fluid interface component) to a sterile, multi-well dish or vessel and exposed to a secondary set of conditions. For example, to be exposed to for 30-40 minutes at 37° C. The cells are suspended in cell medium and either cultured for immediate use or cryopreserved.

The system may use magnetic bead or microfluidic pillar affinity separation to enrich selected cell types for electroporation and transfection. The input is white blood cells collected from the patient and the output is to a conventional cell culture bag for amplification.

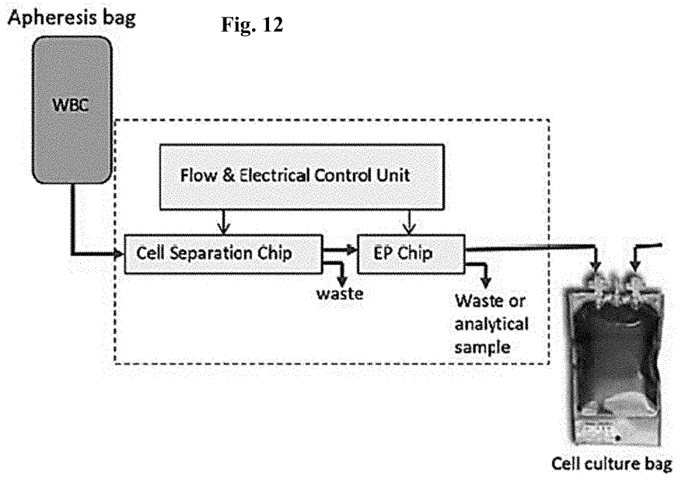

Example process and components are shown in FIG. 12.

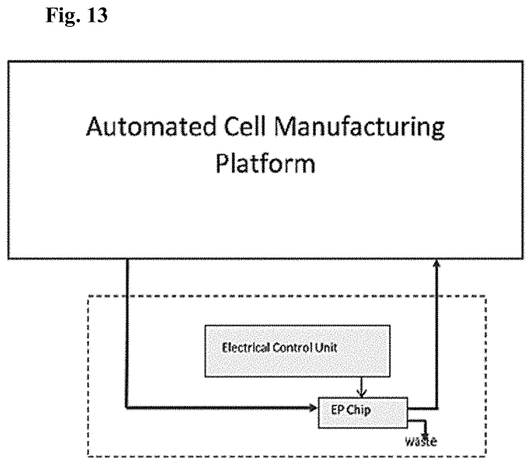

Additionally, the parts or portions of the process may be connected to other processing equipment or stages of a process. For example, in FIG. 13 the electroporation device and components may be implemented with more traditional cell processing hardware.

There is a need for improved methods for selecting the best receptor molecules with which to modify immune system cells for developing treatments for individualized and precisely targeted immunotherapies. Using a flow-based electroporation system it is possible to change the material that one provides to the inputs in time so that one can create many differing combinations of cells and inserted molecules for the purpose of creating libraries of candidate cell treatments for diseases, such as cancer. By selection from this library physicians can select the best type of modified cell for treating a specific person's disease. In addition, our multi-input flow-bases electroporation system can be used to create or manufacture specifically designed combinations of cells with differing chemical modifications as prescribed for an individual patient. In this way, the disclosed methods can facilitate a highly individualized immunotherapy-based treatment of many differing types of cancer and disease.

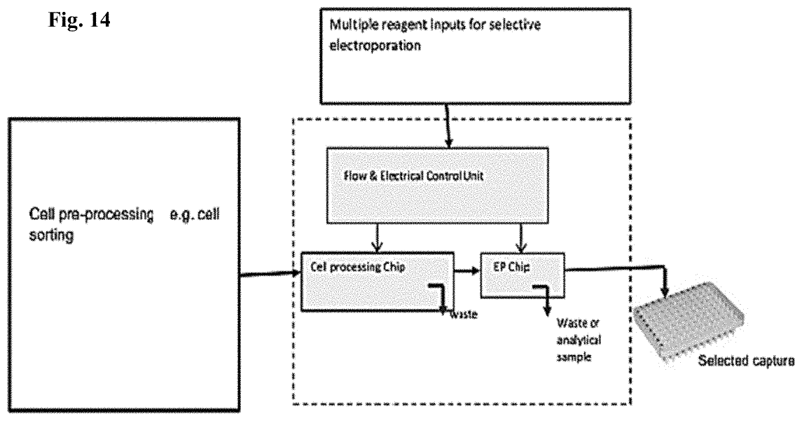

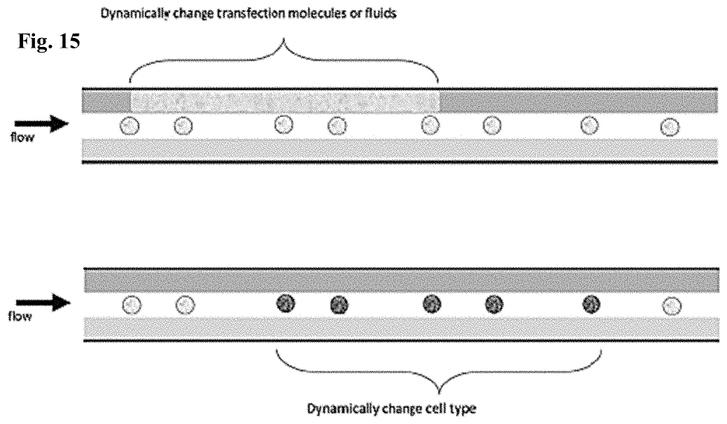

Shown in FIG. 14 is a configuration of a versatile system for rapid controlled processing of cells in the electroporation device. There are numerous advantages that arise from the ability to control and vary the multiple fluid inputs during the electroporation process. By changing the composition of the various fluid streams as a function of time during the electroporation, and collecting the resulting material into different collection vessels, as shown in FIG. 14, it is possible to collect and evaluate a range of electroporation conditions to determine, for example, the best conditions for transfection of a particular cell type or to change the molecules in the stream to evaluate a range of biomolecules to determine which could be valuable for therapeutic use. Such a combinatorial process could greatly reduce the time required to test new compounds and develop cell-based therapies. FIG. 15 indicates that the chemical properties or contents of the fluid streams can be independently varied and controlled to controllably vary the electroporation conditions. Chemical compositions may include, but are not limited to buffers, salts, acids, bases, carbohydrates, peptides, proteins, lipids, and small drug molecules.

Another advantage of this capability is to enable handling small volumes of fluid or small samples of cells and reagents for research. This is particularly valuable in research where the cells may be rare, or the biomolecules may be rare or precious. Such volumes include picoliter, nanoliter, microliter, and milliliter volumes.

Independently controlling the composition of the fluid streams includes possible microfluidic integration of functions for pre and post processing. FIG. 11 shows fluid inputs at different positions along the flow channel. This enables, for example, different chemical conditions to be created before, during, and/or after electroporation. This can be done on the same device (e.g., chip) as show in FIG. 11, or on separate but connected chips as shown in FIG. 12. An important example of this capability includes treatment or selection of cells before electroporation.

Another important application is to maintain different chemical conditions for the cells before, during, or after electroporation. This is valuable, for example, because the conditions for effective electroporation and molecular transfer to the cell may be unhealthy or undesirable for the cells in the longer term. Therefore, changing the chemical composition of the fluid following electroporation enables changing the fluid conditions for the cells immediately after electroporation. This can be done, for example, by flowing in varying chemical-containing solutions such as a nutrient containing medium in which the cells can be maintained effectively for longer times. This nutrient medium can simply be introduced downstream of the electroporation region to dilute the fluid used during the electroporation and establish conditions in which the cells can remain viable or functional for a longer time.

There are numerous other capabilities for enhancing the value of electroporated samples by efficiently processing the material or maintaining different chemical conditions before or after the electroporation process in such integrated systems.

Shown in FIG. 15 is a diagram illustrating two possibilities for changing of input material in the flow streams to create selected and varying combinations of cells and reagents, to be combined to create modified cells.

### Heterologous Objects

In certain aspects, the methods and devices described herein are used to introduce a heterologous object into a cell. The heterologous object can be any object that is small enough to be encompassed by a cell (e.g., small enough to pass through the temporary pore created by electroporation). Such an object can be a nucleic acid (e.g., DNA, RNA), a protein, a peptide, a peptidomimetic, a bead, a dye, a chemical compound, and/or any object that is known in the art to have been introduced into a cell.

In some embodiments, the heterologous object is a nucleic acid. In some embodiments, the nucleic acid is DNA. In other embodiments, the nucleic acid is RNA. In some embodiments, RNA may comprise e.g., mRNA, RNP, small RNA (e.g., siRNA, miRNA, piRNA), RNAi agent, CRISPR/Cas agent (e.g., gRNA).

In some embodiments, the heterologous object modulates gene expression or modulates/alters the genome of a cell (e.g., creates a double-strand break, introduces into the genome a deletion, a substitution, an addition, a mutation (or corrects a mutation present in the genome), or a combination thereof). Systems for altering the genome (e.g., genomic sequence) is well known in the art. Non-limiting examples are provided below.

### CRISPR/CAS

It is art-recognized that CRISPR/Cas system is effective in altering the genome. CRISPR/Cas systems are found in 40% of bacteria and 90% of archaea and differ in the complexities of their systems. See, e.g., U.S. Pat. No. 8,697,359 (incorporated by reference). The CRISPR loci (clustered regularly interspaced short palindromic repeat) are regions within the organism's genome where short segments of foreign DNA are integrated between short repeat palindromic sequences. These loci are transcribed and the RNA transcripts (“pre-crRNA”) are processed into short CRISPR RNAs (crRNAs). There are three types of CRISPR/Cas systems which all incorporate these RNAs and proteins known as “Cas” proteins (CRISPR associated). Types I and III both have Cas endonucleases that process the pre-crRNAs, that, when fully processed into crRNAs, assemble a multi-Cas protein complex that is capable of cleaving nucleic acids that are complementary to the crRNA.

In type II systems, crRNAs are produced using a different mechanism where a trans-activating RNA (tracrRNA) complementary to repeat sequences in the pre-crRNA, triggers processing by a double strand-specific RNase III in the presence of the Cas9 protein or a variant thereof. Cas9 is then able to cleave a target DNA that is complementary to the mature crRNA however cleavage by Cas9 is dependent both upon base-pairing between the crRNA and the target DNA, and on the presence of a short motif in the crRNA referred to as the PAM sequence (protospacer adjacent motif) (see Qi et al (2013) Cell 152: 1173). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The variants of Cas9 are art-recognized, e.g., Cas9 nickase mutant that reduces off-target activity (see e.g., Ran et al. (2014) Cell 154(6): 1380-1389), nCas, Cas9-D10A.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). Thus, exogenously introduced CRISPR endonuclease (e.g., Cas9 or a variant thereof) and a guide RNA (e.g., sgRNA or gRNA) can induce a DNA break at a specific locus within the genome of a target cell. Non-limiting examples of single-guide RNA or guide RNA (sgRNA or gRNA) sequences suitable for targeting are shown in Table 1 in US Application 2015/0056705, which is incorporated herein in its entirety by reference.

In some embodiments, the gene editing nucleic acid sequence encodes a gene editing nucleic acid molecule selected from the group consisting of: a sequence specific nuclease, one or more guide RNA (gRNA), CRISPR/Cas, a ribonucleoprotein (RNP) or any combination thereof. In some embodiments, the sequence-specific nuclease comprises: a TAL-nuclease, a zinc-finger nuclease (ZFN), a meganuclease, a megaTAL, or an RNA guide endonuclease of a CRISPR/Cas system (e.g., Cas proteins e.g. CAS 1-9, Csy, Cse, Cpfl, Cmr, Csx, Csf, cpfl, nCAS, or others). These gene editing systems are known to those of skill in the art, See for example, TALENS described in International Patent Publication No. WO 2013/163628, and U.S. Patent Publication No. 2017/0191078 which are incorporated by reference in their entirety. CRISPR cas9 systems are known in the art and described in U.S. Pat. No. 10,266,850 filed on March 2013, and U.S. Pat. Nos. 8,697,359, 8,771,945, 8,795,965, 8,865,406, 8,871,445 (all of which are incorporated by reference). The devices and methods described herein are also useful for introducing into a cell the deactivated nuclease systems, such as CRISPRi or CRISPRa dCas systems, nCas, or Cas13 systems.

Guide RNAS (gRNAS)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds to a target sequence and e.g., a CRISPR associated protein that can form a ribonucleoprotein (RNP), for example, a CRISPR/Cas complex.

In some embodiments, the guide RNA (gRNA) sequence comprises a targeting sequence that directs the gRNA sequence to a desired site in the genome, is fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq.

A guide sequence can be selected to target any target sequence. In some embodiments, the guide RNA can be complementary to either strand of the targeted DNA sequence. It is appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer et al. “E-CRISP: fast CRISPR target site identification” Nat. Methods 11:122-123 (2014); Bae et al. “Cas-OFFinder: a fast and versatile algorithm that searches for potential off-target sites of Cas9 RNA-guided endonucleases” Bioinformatics 30(10): 1473-1475 (2014); Aach et al. “CasFinder: Flexible algorithm for identifying specific Cas9 targets in genomes” BioRxiv (2014)).

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the single transcript further includes a transcription termination sequence, such as a polyT sequence, for example six T nucleotides. In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a nucleic acid or a composition thereof comprises a nucleic acid that encodes at least 1 gRNA. For example, the second polynucleotide sequence may encode between 1 gRNA and 50 gRNAs, or any integer from 1-50. Each of the polynucleotide sequences encoding the different gRNAs can be operably linked to a promoter. In some embodiments, the promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some embodiments, a nucleic acid for integration into an endogenous locus is introduced in conjunction with another nucleic acid that encodes a Cas nickase (nCas; e.g., Cas9 nickase or Cas9-D10A). It is contemplated herein that such an nCas enzyme is used in conjunction with a guide RNA that comprises homology to an endogenous locus and can be used, for example, to release physically constrained sequences or to provide torsional release. Releasing physically constrained sequences can, for example, “unwind” the nucleic acid such that a homology directed repair (HDR) template homology arm(s) are exposed for interaction with the genomic sequence.

In some embodiments, zinc finger nuclease is used to induce a DNA break that facilitates integration of the desired nucleic acid. “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In some embodiments, a nucleic acid for integration described herein is integrated into a target genome in a nuclease-free homology-dependent repair systems, e.g., as described in Porro et al., Promoterless gene targeting without nucleases rescues lethality of a Crigler-Najjar syndrome mouse model, EMBO Molecular Medicine, (2017). In some embodiments, the in vivo gene targeting approaches are suitable for the insertion of a donor sequence, without the use of nucleases. In some embodiments, the donor sequence may be promoterless.

In some embodiments, the nuclease located between the restriction sites can be a RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to a selected target DNA sequence.

### CRISPR/CAS9 and Variants

As art-recognized and described above, a CRISPR-CAS9 system includes a combination of protein and ribonucleic acid (“RNA”) that can alter the genetic sequence of an organism (see, e.g., US publication 2014/0170753; incorporated by reference). CRISPR-Cas9 provides a set of tools for Cas9-mediated genome editing via nonhomologous end joining (NHEJ) or homologous recombination in mammalian cells. One of ordinary skill in the art may select between a number of known CRISPR systems such as Type I, Type II, and Type III. In some embodiments, a nucleic acid can be designed to include the sequences encoding one or more components of these systems such as the guide RNA, tracrRNA, or Cas (e.g., Cas9 or a variant thereof). In certain embodiments, a single promoter drives expression of a guide sequence and tracrRNA, and a separate promoter drives Cas (e.g., Cas9 or a variant thereof) expression. One of skill in the art will appreciate that certain Cas nucleases require the presence of a protospacer adjacent motif (PAM) adjacent to a target nucleic acid sequence.

RNA-guided nucleases including Cas (e.g., Cas9 or a variant thereof) are suitable for initiating and/or facilitating the integration of a nucleic acid delivered using the devices and methods described herein. The guide RNAs can be directed to the same strand of DNA or the complementary strand.

In some embodiments, the methods and compositions described herein can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9 or a variant thereof) that cannot generate a double strand break (DSB). This permits the endonuclease, in combination with the guide RNAs, to bind specifically to a target sequence in the genome and provide RNA-directed reversible transcriptional control.

Accordingly, in some embodiments, the nucleic acid compositions can comprise a deactivated endonuclease, e.g., RNA-guided endonuclease and/or Cas9 or a variant thereof, wherein the deactivated endonuclease lacks endonuclease activity, but retains the ability to bind DNA in a site-specific manner, e.g., in combination with one or more guide RNAs and/or sgRNAs. In some embodiments, the nucleic acid can further comprise one or more tracrRNAs, guide RNAs, or sgRNAs. In some embodiments, the de-activated endonuclease can further comprise a transcriptional activation domain.

In some embodiments, the nucleic acid compositions for integration of a nucleic acid of interest into an endogenous locus can comprise a hybrid recombinase. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases include those described in Gaj et al. Enhancing the Specificity of Recombinase-Mediated Genome Engineering through Dimer Interface Redesign, Journal of the American Chemical Society, (2014).

The nucleases described herein can be altered, e.g., engineered to design sequence specific nuclease (see, e.g., U.S. Pat. No. 8,021,867; incorporated by reference). Nucleases can be designed using the methods described in e.g., Certo et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; and 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

### MegaTALs

In some embodiments, the nuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42(4):2591-601 and Boissel 2015 Methods Mol Biol 1239: 171-196. Protocols for megaTAL-mediated gene knockout and gene editing are known in the art, see, e.g., Sather et al. Science Translational Medicine 2015 7(307):ra156 and Boissel et al. 2014 Nucleic Acids Research 42(4):2591-601. MegaTALs can be used as an alternative endonuclease in any of the methods and compositions described herein.

### CAR Molecules and CAR Therapy

The devices and methods of the present disclosure provide a particular utility for introducing a heterologous object into a cell for a CAR therapy. Chimeric antigen receptors (CARs) are transmembrane proteins that have been engineered to give the cells (e.g., T cells, macrophages, NK cells) the new ability to target/bind a specific protein. The receptors are chimeric because they combine both antigen-binding and certain cellular functions (e.g., T cell activating function) into a single receptor. For example, the receptor can comprise an extracellular antigen-binding domain (e.g., scFv) that binds to a specific antigen (e.g., those highly and specifically expressed on the surface of cancer cells) fused to a transmembrane domain and an intracellular costimulatory domain/activation domain.

### CAR T Therapy

Chimeric antigen receptor T cells (CAR T cells) are T cells that are engineered to express the CAR proteins for cancer therapy. CARs enable T cells to recognize tumor-associated antigens (TAAs) in a major histocompatibility complex (MHC)-independent manner. CAR T therapy can use T cells that are autologous or allogeneic to the patient. After CAR T cells are infused into a patient, they act as a “living drug” against cancer cells. When they come in contact with their targeted antigen on a cell, CAR T cells bind to it and become activated, then proceed to proliferate and become cytotoxic. CAR T cells destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors. The first CAR T cell therapies were FDA-approved in 2017, and there are now 6 approved CART therapies.

There are several variations/generations of CAR designs. The first reports of tumor-targeting CARs demonstrated that an scFv recognizing antigens such as human epidermal growth factor receptor 2 (HER2) fused to the CD3ζ signaling domain can elicit tumor-specific cytotoxicity, but T cells expressing these “first-generation” CARs that included only the CD3ζ chain for T-cell signaling generally failed to elicit potent antitumor effects. In the following years, second- and third-generation CARs emerged that included one or two costimulatory domains, respectively, drawing from the biological understanding that the endogenous TCR requires association with other costimulatory or accessory molecules for robust signaling. Most commonly derived from CD28 or 4-1BB, these costimulatory domains conferred more potent antitumor cytotoxicity, increased cytokine production, and improved proliferation and persistence of CAR-T cells. The choice of costimulatory domain has an impact on a wide range of properties, including metabolic pathways, T-cell memory development, and antigen-independent tonic signaling, prompting further research into other costimulatory domains. For example, a third-generation CAR with OX40 and CD28 costimulatory domains repressed CD28-induced secretion of interleukin (IL)-10, an anti-inflammatory cytokine that compromises T-cell activity. In addition, the inducible T cell costimulator (ICOS) costimulatory domain in combination with either CD28 or 4-1BB costimulation increased in vivo persistence and MyD88/CD40 costimulation improved in vivo proliferation of CAR-T cells. More recently, fourth-generation CARs that incorporate additional stimulatory domains, commonly referred to as “armored” CARs, have been reported. In one example, the engineered armored CAR-T cells termed “T cells redirected for universal cytokine-mediated killing” (TRUCK) have been engineered to secrete the proinflammatory cytokine IL-12 to stimulate innate immune cells against the tumor and resist inhibitory elements of the TME, including regulatory T (Treg) cells and myeloid-derived suppressor cells (MDSCs). The secretion of other soluble factors has been studied, including IL-15 or IL-18 to enhance T cell proliferation, as well as the combination of CCL19 and IL-7 to recruit endogenous immune cells and establish a memory response against tumors.

The devices and methods of the present disclosure can be used in introducing a nucleic acid to a T cell for generations of the CAR T cells for use as e.g., a cancer therapy.

### Dual Car Therapy

CAR T cells with ability to target two antigens on a cancer cell surface have been proven to be effective clinically. For example, CART cells with dual targeting of CD19 and CD22 in adult patients with recurrent or refractory B cell malignancies showed improved efficacy (Spiegel et al. (2021) Nature Medicine, 27:1419-1431).

In addition, dual CAR T demonstrated effectiveness in targeting tumor cells with heterogeneous antigen expression. For example, CAR-T cells targeting simultaneously two tumor-associated antigens with trans-acting CD28 and 4-1BB co-stimulation caused rapid antitumor effects in in vivo stress conditions, protection from tumor re-challenge and prevention of tumor escape due to low antigen density. Molecular and signaling studies indicated that T cells engineered with the dual CAR design demonstrated sustained phosphorylation of T-cell-receptor-associated signaling molecules and a molecular signature supporting CAR-T-cell proliferation and long-term survival. Furthermore, metabolic profiling of CAR-T cells displayed induction of glycolysis that sustains rapid effector T-cell function, but also preservation of oxidative functions, which are critical for T-cell long-term persistence (Hirabayashi et al. (2021) Nature Cancer, 2:904-918).

### CAR-M Therapy

Programming CARs into cell types other than T cells can further expand the versatility of the therapy by realizing new functions unachievable by CAR T cells. It was recently demonstrated that primary macrophages can be engineered with CARS via adenoviral transduction (Klinchinsky et al. (2020) Nat Biotechnol). The resulting CAR M cells exhibited tumor-specific phagocytosis, inflammatory cytokine production, polarization of bystander macrophages to the immunostimulatory M1 phenotype, and cross-presentation of the tumor associated antigen (TAA) to bystander T cells.

### CAR-NK Therapy

CD19-targeting CAR-NK cells have achieved robust clinical efficacy without inducing cytokine release syndrome (CRS), neurotoxicity, or graft-versus-host syndrome (GvHD) in patients with B-cell lymphoid tumors. CAR NK cells have been shown to exert potent and specific cytotoxicity toward a variety of tumor models, including leukemia, multiple myeloma, ovarian cancer, and glioblastoma, as well as toward immunosuppressive cell types such as myeloid-derived suppressor cells (MDSCs) and follicular helper T cells (TFH). Lastly, natural killer T (NKT) cells possess antitumor and tumor-homing capabilities, and GD2-targeting CAR NKT cells that harness these inherent advantages exhibited effective localization to and lysis of neuroblastoma cells without significant toxicity (Xu et al., (2019) Clin Cancer Res 25:7126-7138).

### Exemplary Embodiments

1. A flow device for electroporation, comprising

at least one planar flow channel;

at least one pair of electrodes;

at least one first port; and

at least one second port.

2. The flow device of 1, wherein the at least one second port is located:

(a) on the bottom of the flow device (e.g., FIG. 4A) such that the flow from the second port is perpendicular to the flow within the channel; or

(b) on the side of the flow device (e.g., FIG. 4B) such that the flow from the second port is in the same direction as the flow within the channel.

3. The flow device of 1 or 2, wherein the width of the device is at least about 1, 2, 3, 4, 5, 6, 7, or 8 mm but no more than about 9, 10, 11, 12, 13, 14, 15, 16, 17, 18, 19, or 20 mm.
4. The flow device of any one of 1-3, wherein the width of the device is about 8 mm.
5. The flow device of any one of 1-4, wherein the width of the channel is at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, or 7 mm.
6. The flow device of any one of 1-5, wherein the width of the channel is at least about 2 mm but no more than about 7 mm, optionally wherein the width of the channel is about 2 mm.
7. The flow device of any one of 1-6, wherein the at least one pair of electrodes extend to at least one edge of the flow device, optionally distal to the at least one second port.
8. The flow device of any one of 1-7, wherein the at least one first port is located on the bottom of the flow device, on top of the flow device, on the side of the flow device, or any combination thereof.
9. A microfluidic flow-based electroporation system, comprising

at least one flow device of any one of 1-8;

at least one electrical control module that connects to the pair of electrodes;

at least one fluid delivery system that links to the flow device at the at least one first port; and

a multi-well module that links to the flow device at the at least one second port.

10. The system of 9, wherein the system comprises at least 1, 2, 3, 4, 5, 6, 7, 8, 9, 10, 11, 12, 13, 14, 15, or 16 flow devices.
11. The system of 9 or 10, wherein the system comprises 8 flow devices.
12. The system of any one of 9-11, wherein the flow device is positioned horizontally or vertically with respect to the multi-well module.
13. The system of any one of 9-12, wherein the multi-well module comprises a 6-, 24-, 48-, 96-, or 384-well plate.
14. The system of any one of 9-13, wherein the at least one pair of electrodes is connected to the at least one electrical control module from the top, bottom, or the side of the flow device.
15. The system of any one of 9-14, wherein the fluid delivery module allows moving fluid from a first port toward a second port, from a second port toward a first port, or from either port toward the other port.
16. The system of any one of 9-15, wherein the fluid delivery module comprises a syringe or a pump.
17. The system of any one of 9-16, wherein a fluid delivery module connects to a flow device at a first port via a tubing; and/or a multi-well module connects to a second port via a tubing.
18. The system of any one of 9-17, further comprising a robotic module that moves the multi-well module relative to the flow device.
19. The system of any one of 9-17, further comprising a robotic module that moves the flow device relative to the multi-well module.
20. The system of 18 or 19, wherein the robotic module is selected from any one of the robotic modules listed in Table 1.
21. The system of any one of 9-20, wherein the at least one electrical control module provides a voltage that has a bipolar square wave, a dual voltage waveform, periodic waveform, or a periodic arbitrary time-varying voltage.
22. The system of any one of 9-21, wherein the at least one electrical control module provides a voltage that has a period waveform.
23. The system of 22, wherein:

(a) the periodic waveform is a sinusoidal function of time, wherein the sinusoidal function has an absolute amplitude from zero that is at most 200 Volts, a frequency that is at least 10 Hz and at most 100 kHz, and a phase that is at least 0 and at most 2n;

(b) the periodic waveform has a first frequency and a second frequency different from the first frequency;

(c) the periodic waveform is a Fourier series; and/or

(d) the periodic waveform is a square waveform or a rectangular waveform having a voltage amplitude of at least 0.1 V and at most 100 V, and a frequency of at least 100 Hz and at most 1 THz.

24. The system of 23, wherein:

(a) the square waveform or a rectangular waveform is bipolar; and/or

(b) the square waveform or a rectangular waveform further comprises a direct current component of at most ±10 V.

25. The system of any one of 9-24, wherein the at least one electrical control module

(a) is connected to the at least one pair of electrodes independently from any other pair of electrodes; and/or

(b) allows forming an electric field as a function of time and/or position within the fluid channel.

26. The system of any one of 9-25, wherein the flow device comprises at least two first ports and at least two fluid delivery systems, wherein each fluid delivery system is connected to a different first port.
27. The system of 26, wherein the fluid delivery system allows modulating the flow rate and chemical composition in one of the at least two streams as a predetermined function of time, position, or both time and position independently from any other fluid stream within the channel.
28. The system of any one of 9-27, further comprising:

(a) a flow sensor;

(b) a flow-rate control module;

(c) a temperature control module;

(d) a fluid interface that couples the fluid delivery system to the flow device;

(e) a cell processing module;

(f) an electrical or optical monitoring module coupled to the flow device; or

(g) any combination of two or more of (a)-(f).

29. The system of 28, wherein the cell processing module is upstream from the flow device.
30. The system of any one of 9-29, wherein the cell processing module allows cell sorting, selection, labeling, analysis, or a combination thereof.
31. The system of any one of 9-30, wherein the cell processing module comprises a fluorescence-activated cell sorting component.
32. The system of any one of 9-31, wherein the cell processing module comprises a magnetic field source that allows magnetic bead separation.
33. The system of any one of 9-32, wherein the cell processing module is built in the device (e.g., FIG. 11) or built in another microfluidic device (e.g., FIG. 12).
34. The system of any one of 9-33, further comprising an apheresis bag upstream of the cell processing module.
35. A method of electroporating a cell, comprising flowing a cell through the flow device of any one of 1-8 or the system of any one of 9-34, and applying voltage to the electrodes.
36. A method of identifying at least one suitable electroporation parameter, the method comprising
(a) electroporating cells according to the method of 35, or using the flow device of any one of 1-8 or the system of any one of 9-35, under at least one parameter;
(b) electroporating cells under at least one additional parameter;
(c) comparing the transfection efficiency, cell viability, or both of the cells in (a) and (b); and
(d) identifying an electroporation parameter that yields a higher transfection efficiency, cell viability, or both as a suitable electroporation parameter.
   37. The method of 36, wherein the at least one electroporation parameter comprises cell type, cell concentration, heterologous object type, heterologous object concentration, fluid flow rate, fluid volume, buffer type, time-dependent voltage waveform, collection time, or a combination of two or more thereof.
   38. The method of 36 or 37, wherein the at least one electroporation parameter comprises cell type, cell concentration, heterologous object type, heterologous object concentration, fluid flow rate, fluid volume, buffer type, time-dependent voltage waveform, and collection time.
   39. The method of 37, wherein the cell type, cell concentration, heterologous object type, heterologous object concentration, fluid flow rate, fluid volume, buffer type, or a combination of two or more thereof is kept constant.
   40. The method of any one of 36-39, wherein cells are electroporated using a system comprising at least 2 flow devices, optionally 8 flow devices.
   41. The method of any one of 36-40, wherein the cells are electroporated using a system comprising a multi-well module, optionally wherein the multi-well module comprises a 98-well plate.
   42. The method of any one of 36-41, wherein the cells are electroporated using a system comprising a robotic module.
   43. The method of any one of 35-42, wherein the method identifies a suitable electroporation parameter for manufacturing cells for cellular therapies.
   44. The method of 43, wherein the cellular therapies comprise a CAR therapy.
   45. The method of any one of 35-44, wherein the method electroporates cells with a heterologous object, optionally a mammalian cell or a human cell.
   46. The method of 45, wherein the cell is a lymphocyte.
   47. The method of any one of 35-46, wherein the cell is a T cell, optionally a primary T cell.
   48. The method of any one of 37-47, wherein the heterologous object comprises a nucleic acid.
   49. The method of any one of 37-48, wherein the heterologous object comprises an mRNA.
   50. The method of any one of 37-49, wherein the heterologous object comprises a CRISPR/Cas9 RNP.
   51. The method of any one of 35-49, wherein the method modifies a genome of the cell.

### EXAMPLES

The disclosure will be further illustrated with reference to the following specific examples. These examples are given by way of illustration and are not meant to limit the disclosure or the claims that follow.

### Example 1: Materials & Methods

### Fabrication of Electroporation Flow Chips

Electroporation flow chips were constructed from a three-layer stack of polymer substrates. All three layers were laser cut with a small beam spot, high resolution CO₂ laser. The top and bottom layers, cut from 1 mm thick acrylic slabs (McMaster Carr, Robbinsville, N.J., USA), create the floor and sealing channel surfaces. The middle layer was a spacer that defines the channel depth and width and composed of a thin (typically 80 μm) polymer film with medical adhesive on each side (Adhesives Research, Glen Rock, Pa., USA). To fabricate the chip, the bottom and top acrylic layers were laser-cut into 1″×2″ pieces. The pieces were then laser-cut to add fluid inlet/outlet ports and alignment holes for use during the assembly process. Afterwards, a thin film electrode of gold was deposited by physical vapor deposition using a CVC SC4500 electron-gun evaporation system on the inside surface of each acrylic piece at the Cornell NanoScale Facility (CNF) using a shadow mask. The middle layer was cut to shape and also received the corresponding alignment holes via the laser cutting process. The three-piece (acrylic, polymer film, acrylic) sandwich assembly was then compression bonded in a press.

### Cell Culture and Reagents

Primary T cells were purchased from StemCell Technologies and cultured in animal-product free ImmunoCult media supplemented with 100 μg/mL recombinant human IL-2 (StemCell, Vancouver, BC, Canada). Primary T cells were thawed, permitted to rest overnight in media, and subsequently activated with CD3/CD28 antibodies (StemCell) per manufacturer's instructions. Two days post-activation, cells were harvested for transfection. Jurkat cells were purchased from Millipore Sigma (Millipore Sigma, Burlington, Mass., USA) and cultured in RPMI 1640 medium supplemented with 10% fetal bovine serum (R&D Systems, Minneapolis, Minn., USA). All cells were maintained at 37° C. and 5% CO₂.

DNA, mRNA, and CRISPR/Cas9 Constructs

pCMV-GFP (plasmid DNA; 3705 bp) was purchased from Altogen Biosystems (Las Vegas, Nev., USA). Dasher GFP mRNA (1 kb) was purchased from Aldevron. SpCas9 and sgRNA targeting the TRAC locus (AGAGUCUCUCAGCUGGUACA) were purchased from Synthego (Synthego, Pasadena, Calif., USA). Ribonucleoprotein (RNP) complexes were formed by mixing equimolar quantities of SpCas9 and sgRNA and incubating for 10 minutes at room temperature before addition to cell suspensions in electroporation buffer.

### Flow Cytometry

Primary T or Jurkat cells were withdrawn 24-h (GFP expression) or 72-h (TCR-α) after transfection for flow cytometry analysis using a ZE5 Cell Analyzer (Bio-Rad, Hercules, Calif., USA). TCR-α expression was measured by staining primary T cells using AlexaFluor 488 anti-human TCR-α antibody (BioLegend, San Diego, Calif., USA). Viability was measured by staining cells with the viability dye 7-AAD and incubating for 5 minutes prior to flow analysis (Fisher Scientific, Hampton, N.H., USA). During flow analysis, cells were first gated to exclude cell debris using forward scatter (FSC) area vs. side scatter (SSC) area plots. Single cells were subsequently gated using FSC-area and FSC-height plots. To measure viability, single cells were gated to measure the percentage of 7-AAD negative (live) and positive (dead) populations. To measure GFP or TCR-α expression, viable cells were gated to measure the percentage of cells with green fluorescence relative to zero-voltage controls.

### Example 2: An Exemplary Device

An exemplary device incorporates a single use, continuous-flow microfluidic channel capable of efficient and reproducible electrotransfection of cells. The microfluidic channel consists of a planar flow chip with a thin slab geometry. Electrodes are patterned on the top and bottom flow surfaces in order to apply a uniform electric field perpendicular to the flow direction. The electric field is applied with a continuously cycling arbitrary voltage waveform. The thin channel height, ranging from 50-100 μm, ensures that each cell is subject to the same electric field and chemical environment to enable reproducible electroporation. The thin channel height also permits us to achieve the necessary electric field strength to transiently open pores in the plasma membrane using relatively low voltage amplitudes (5-30 V) compared to the high voltage of traditional commercial systems. The width (1-10 mm) of the devices used in these experiments is much larger than its depth to allow for rapid and continuous flow of the cells through the chip. Our planar geometry enables optimal transfection parameters to be determined using small volumes and channel widths before scaling up to large volumes and channel widths for clinical-scale delivery.

The exemplary device is designed to integrate with automated cell processing approaches using a selectable, computer-controlled voltage waveform and robotic fraction collection. The device is capable of delivering any arbitrary electrical waveform. During typical operation, cells are driven into the channel by a syringe pump through a single fluid inlet and exit through a single fluid outlet. Cells are typically suspended in a low conductivity electroporation buffer containing the cargo to be delivered. To optimize electrotransfection, a custom MATLAB script controls a function generator containing pre-programmed electrical waveforms while a robotic autosampler is programmed to move the outlet tubing for dispensing in a multi-well plate. Electrical waveform selection and robotic autosampling are programmed such that each well contains a pure population of cells that received one pre-programmed voltage waveform. In this study, we focus on the use of bipolar rectangular waveforms that can be described by their frequency (f), duration (t), and voltage amplitude (V). Overall, this robotic setup enables rapid sweeping of electrical parameters to select conditions desirable for transfection. Larger scale transfection is then performed with the selected electrical conditions.

### Example 3: Electroporation of Mammalian Cells with a DNA Plasmid

This example describes an embodiment where the flow electroporation device is used to electroporate mammalian cells with a DNA plasmid. Chinese hamster ovary (CHO-Kl) cells (ATCC) are electroporated with a plasmid that expresses green fluorescent protein using the flow-through electroporation device described. Cell viability can be determined based on the uptake of propidium iodide. The electroporation efficiency can be determined using fluorescent observation of the number of cells that express the green fluorescent protein relative to the total number of cells.

The cells are cultured in an incubator at 37° C. and 5% CO₂. The cells can be cultured in a synthetic medium, such as Dulbecco's modified Eagle's Minimum Essential Medium (DMEM, Sigma, St. Louis, Mo.) supplemented with 10% fetal bovine serum (Sigma) and 100 mg/mL streptomycin (Sigma). When the cell suspension density reaches a certain value, for example, 2×10⁶ cells/mL, the cell suspension is diluted with additional culture medium. Prior to introduction into the device, a 10 mL sample of the suspension is centrifuged at 300 g for 5 min. The supernatant is discarded, and the cells are re-suspended in a low conductivity buffer (described below). The cell suspension density for electroporation is preferably 1×10⁸ cells/mL with a range between 1×10⁷ and 1×10⁹ cells/mL.

The low conductivity buffer is composed of 0.8 mM Na₂HPO₄, 0.2 mM KH₂PO₄, 0.1 mM MgSO₄.7H₂O, and 250 mM sucrose, at a pH of 7.4. This buffer is made by dissolving 0.1136 g of Na₂HPO₄, 0.0272 g of KH₂PO₄, 0.02465 g of MgSO₄.7H₂O, and 85.575 g of sucrose in 1 liter of water, and subsequent adjustment of the pH. The sucrose is used to equalize the osmotic pressure of the buffer with that of the cells. The buffer is filtered with a 0.2-micron membrane and stored at 4° C. The concentrations of salts in the buffer as described result in a solution with electrical conductivity of approximately 0.014 S/m. The preferable range of the electrical conductivity of this buffer is between 1×10⁻³ and 2.5 S/m.

The pAcGFP-C1 plasmid (4.7 Kb, Clontech, Mountain View, Calif.) encodes a green fluorescent protein (GFP) from Aequorea coerulescens and contains an SV40 origin for replication in mammalian cells. The GFP protein is excited at 475 nm and emits at 505 nm. The plasmid is amplified in E. coli and purified using the QIAfilter Plasmid Mega Kit (Qiagen, Valencia, Calif.) according to the manufacturer's instructions. The plasmid DNA is dissolved in Tris-EDTA buffer and stored at −20° C. until use. The plasmid DNA concentration is determined by ultraviolet (UV) absorbance at 260 nm. Prior to an electroporation experiment, the plasmid is precipitated with ethanol and resuspended in phosphate buffered saline (PBS, 137 mM NaCl, 2.7 mM KCl, 10 mM Na₂HPO₄, 1.8 mM KH₂PO₄) buffer with an electrical conductivity of approximately 1.5 S/m at a concentration of approximately 40 ug/mL. The range of the electrical conductivity of this buffer is between 1×10⁻² and 10 S/m. The range of the plasmid concentration is between 0.01 and 100 ug/mL.

The low electrical conductivity buffer used for the cell flow inlet used in combination with a higher electrical conductivity buffer (PBS) for the upper and lower sheath inlets and flow layers results in an electric field that is substantially larger across the cell flow layer for a given applied voltage. For a typical experiment, the pressure of each flow is adjusted so that the cell flow layer is approximately 50 microns deep and the upper and lower sheath flow layers are approximately 25 microns each in depth. The electrical conductivities of the low and high conductivity buffer are 0.014 S/m and 1.5 S/m, respectively. The electrical resistance of the sheath layer (for a voltage applied between the two support block surfaces) is approximately 99% of the total resistance. This means that if 5 V is applied between the electrodes on the two support blocks that the electric fields in the streams adjacent to the electrodes is approximately 9 V/cm while the stream sandwiched between those two streams is 991 V/cm.

It is known that a difference of approximately 1 V between the interior and exterior of a certain cell will result in the formation of pores that can allow for the passage of nucleic acid molecules. The potential difference U across a cell membrane at a point on the surface of a cell in an external electric field of strength E is given by U=f ER cos θ, where R is the cell radius, θ is the angle between the electric field and the normal to the cell surface, and f is a geometric factor that is typically around 3/2. This implies that to form pores at the poles of the cell the electric field should be about 1 kV/cm for a cell with radius of 8 microns.

With this electroporation device, the application of a 5 V potential difference between the top and bottom plates results in an electric field within the cell flow layer of about 1 kV/cm given the electric field strengths and flow layer depths described. The preferable range of applied voltages is between 1 V and 100 V. If the patterned electrodes are 2.5 cm by 0.5 cm in size, then for a 5 V applied potential, a current of about 0.17 A is generated and a power of 0.87 J/s is dissipated. This amount of power would increase the temperature of pure water in a device with dimensions 5 cm by 2.5 cm by 0.01 cm by 1.7 degrees C./s, assuming that no heat is dissipated through the boundary. The source for the applied voltage can be from a battery with a fixed voltage or a battery used in conjunction with a resistive voltage divider to enable the voltage to be varied over the selected range. Commercial voltage supplies are also readily available to provide selected voltages in the range of 1 V to 100 V. An alternative electrode size example includes electrodes with dimensions of 2.5 cm by 0.05 cm in size, then for a 5 V applied potential, a current of about 0.017 A is generated and a power of 0.087 J/s is dissipated. This amount of power would increase the temperature of pure water in a device with dimensions 5 cm by 2.5 cm by 0.01 cm by 0.17 degrees C./s. In a typical experiment, cells at a density of 1.0×10⁷/mL are flowed through the device (e.g., chip) at a volumetric rate of approximately 1.5 mL/min, with a preferable range between 0.01 and 100 mL/min. The nominal flow rate of 1.5 mL/min results in an average linear flow velocity of 1.0 cm/s. At this velocity, cells are subject to the electric field from an electrode that is 2.5 cm by 0.5 cm in width and length for 0.5 s. Assuming Hele-Shaw flow, the pressure difference across the input and output of the device (e.g., chip) is about 40 atm. It is important to note that during the approximately 0.5 s that cells are subject to the electric field, that the plasmid DNA is electrophoretically driven toward the cell flow layer, assuming that the plasmid is in the lower sheath flow and that the top electrode is held at a higher voltage than the bottom electrode. Assuming a DNA mobility of 4×10⁴ cm²/Vs, the average time that it takes a DNA molecule to move half-way through a distance of 25 microns (the typical depth of the sheath flow layer containing the plasmid) is 0.34 s. A DNA molecule that reaches the cell flow layer is driven across it in about 10 ms.

Another important timescale is the cell sedimentation time for falling a distance of one-half of the cell flow layer thickness. Again assuming a cell radius of 8 microns, a difference in density between a cell and the surrounding fluid of 0.07 g/cm³, and that the hydrodynamic friction coefficient of a cell is 6πηR, where η is the buffer viscosity (approximately 0.001 Pa, but may be higher with additive chemicals such as sucrose), the time to drop a distance of 25 microns is approximately 0.4 s. And the time for a typical salt ion, such as Na or K, to diffuse a distance of 25 microns is 0.6 s. This indicates that the flow layers remain laminar (and retain their respective conductivities) for the time it takes the cells to cross the electrode region when the patterned electrodes are about 2.5 cm by 0.5 cm in width and length.

Following the electroporation of a given volume of cells the electroporation efficiency and cell viability are determined by phase contrast and static fluorescent imaging, and sometimes by flow cytometry. After the cells are electroporated with the GFP-expressing plasmid in the flow device (e.g., chip), the cells are collected and transferred to a 96 or 24 well plate with appropriate cell medium, such as DMEM. The cells are cultured at 37° C. in an incubator with 5% CO₂ for 1, 6, 12, 24, or 48 hours. The cells are centrifuged at 300 g for 5 min and the aspirant is discarded. The cells are washed with PBS and this process is repeated. Following this, the cells are stained with propidium iodide (Invitrogen) at a concentration of approximately 1 microgram/mL. The cells are incubated in the dark for 15 min and then optically examined by phase contrast under fluorescent filters. A standard GFP filter set is used to determine the fraction of cells that have been electroporated with the plasmid. A filter set with excitation at 488 nm and emission at approximately 620 nm is used to determine the dead cells that have been permeated by propidium iodide. Several images can be acquired at different locations to improve the statistics of the electroporation efficiency and the cell viability. The cells may also be examined by flow cytometry to determine the fraction that has been electroporated as identified by a green fluorescent signal and the fraction that are dead as identified by uptake of propidium iodide and a red fluorescent signal.

Thus, the described device can reliably be used to electroporate a large number of mammalian or bacterial cells at high efficiency and with low cell death in a short amount of time. The cells can be transfected with plasmid DNA that can be transcribed into a protein that is therapeutic for a disease. The cells can be transfected with mRNA that is likewise transcribed into a protein that is necessary for improving the health of the cell or that can be harvested for other medical use, such as production of antibodies. The cells can also be transfected with purified Cas9 protein, or another DNA guided nuclease, and synthetic guide RNA molecules, termed ribonucleoproteins, to efficiently edit a genomic site that is deleterious.

### Example 4: Electroporation of Different Types of Cells and Molecules

The method outlined in Example 3 can be used to electroporate a variety of different mammalian cell types including: CHO, Hela, T-cells, CD8+, CD4+, CD3+, PBMC, Huh-7, Renca, NIH 3T3, Primary Fibroblasts, hMSCs, K562, Vero, HEK 293, A549, B16, BHK-21, C2C12, C6, CaCo-2, CAP-T, COS-1, Cos-7, CV-1, DLD-1, H1299, Hep G2, HOS, Jurkat, L5278Y, LNCaP, MCF7, MDA-MB-231, MDCK, Mesenchymal Stem Cells, Min-6, Neuro2a, NIH3T3L1, NSO, Panc-1, PC12, PC-3, RBL, RLE, SF21, SF9, SH-SY5Y, SK-MES-1, SK—N—SH, SL3, SW403, THP-1, U205, and U937.

The method outlined in Example 3 can be used to electroporate a variety of different types of molecules to any mammalian cell including: DNA, RNA, mRNA, siRNA, miRNA, other nucleic acids, proteins, peptides, enzymes, metabolites, membrane impermeable drugs, cryoprotectants, exogenous organelles, molecular probes, nanoparticles, lipids, carbohydrates, small molecules, and complexes of proteins with nucleic acids (like CAS9-sgRNA). While the method outlined in Example 3 relies on an electric field to deliver charged nucleic acid molecules to electroporated cells, the method also suffices to electroporate neutral molecules where diffusive motion is sufficient for the delivery.

### Example 5: Electroporation Using Time Varying Voltage Waveforms

The method outlined in Example 3 can be used to electroporate cells with a variety of different voltage waveforms applied. Cells can be electroporated when applying a rectangular waveform with a frequency of 10 kHz and a peak to peak voltage difference of approximately 10 V. The preferable range of the peak to peak voltage difference is between 0.1 and 100 V. The preferable range of the frequency is between 100 Hz and 1 THz. The square wave could be bipolar so that the time averaged current is zero. The square wave could also have an additional DC component of preferably less than plus or minus 10 V. The applied waveform could be sinusoidal, saw-tooth, rectangular, triangular, or be a sum of any number of sinusoidal shapes with different frequencies and amplitudes in time.

The method outlined in Example 3 can be used to improve the efficiency of cellular electroporation by the application of different voltage waveforms to different electrode pairs. A first electrode pair could be used to apply a square waveform with a frequency of 10 kHz and a peak to peak voltage difference of approximately 10 V to permeabilize the membranes of the cells passing in the fluid channel. A second or third or more pair of electrodes could be used to apply a DC or oscillating voltage that preferentially directs charged molecules, like nucleic acids, toward the permeabilized cells. The second or third or more pair of electrodes could be used to exert electric forces on the cells or molecules in solution, which creates a relative velocity between the cells and the fluid, the molecules and the fluid, or between the cells and charged or neutral molecules in solution. The preferred range of the voltage amplitude or offset applied by the second or more pair of electrodes is between 1 mV and 100 V. The second or more pair of electrodes could be used to apply electrical forces that are along the direction of flow in the device or perpendicular to the direction of flow. Pairs of electrodes that are used to exert electric forces may be on the same surface or opposite surfaces of the device, where each surface is in contact with the fluid. The second pair of electrodes may be used to permeabilize structures within the cell after the membrane has been porated. The second or more pair of electrodes may be used to apply electric fields that result in the concentration increase of nucleic acid or other molecules at the interface between fluid layers of varying conductivity. The second or more pair of electrodes may be used to apply electric fields that cause the rotation of the cell so more surface area is exposed to the nucleic acid or other molecules in solution.

### Example 6: Electroporation of Cells in a Single Stream of Low Conductivity Buffer

For electroporation experiments, cells were harvested, counted, and washed two times in BTXpress Cytoporation low-conductivity electroporation buffer (Holliston, Mass., USA). After the second wash, cells were resuspended in the same electroporation buffer at a density of 5×10⁶ cells/mL unless otherwise noted. The cargo to be delivered was subsequently added at the indicated concentrations for each experiment. Aqueous solutions containing cells and cargo were then loaded into a syringe, which was then loaded into a syringe pump (Chemyx, Stafford, Tex., USA). Cell suspensions were flowed continuously into the flow cell at 320 μL/min (2-mm channel width) or 1600 μL/min (10-mm channel width) unless otherwise noted. As cells transit under the electrode, they were subjected to the indicated arbitrary voltage waveform generated by a function generator (Siglent SDG 1032X; Siglent, Technologies, Solon, Ohio, USA) and amplified by a RF amplifier (TS250; Accel Instruments, Irvine, Calif., USA). As cells exit the outlet, they are dispensed into wells containing pre-warmed cell media by a custom-built robotic fraction collector.

Throughout each experiment, the function generator and oscilloscope were controlled using a custom MATLAB program to deliver one to ten pre-programmed arbitrary voltage waveforms over the experiment's duration (v. 2021a, Mathworks, Natick, Mass., USA). Waveform switching and the robotic autosampler were programmed to ensure that each well contained a pure population of cells that received one pre-programmed voltage waveform. Voltage waveforms and the voltage across a 1-ohm resistor in series with the flow chip were monitored by an oscilloscope (Siglent SDS1104X-E, Siglent Technologies). Transfection efficiency, cell viability, and relative yield were measured 24-h post-transfection (unless otherwise noted) by flow cytometry.

### Example 7: Buffer Exchange

In some instances it may be desirable for the cells to be transported into the device in one buffer and then to exchange that buffer within the device for another buffer more suitable for electroporation. It may also be advantageous after the cells have been subject to an arbitrary voltage waveform in the electroporation buffer, to exchange the electroporation buffer for a third recovery buffer that enhances the ability of the cells to recover (increases their viability). The first buffer may be cell media or a high conductivity buffer of a suitable pH. The electroporation buffer may be lower conductivity and of a different chemical composition and pH. The third, recovery buffer, may be identical to the first buffer or may also contain additional chemicals that aid in cell recovery.

A simple method to exchange buffers within the fluidic device is to have another inlet and outlet somewhere downstream from the first inlet, most likely on opposite sides of the fluid channel from each other. The second inlet and outlet pair may be directly opposite each other or offset. A pump or fluid control module flows the electroporation buffer into the second inlet. Fluid is directed toward a waste container from the second outlet pair. The fluid in the device at this location is now a mixture of the first buffer and the electroporation buffer. The mixture depends on the input flow of the first buffer, the input flow rate of the electroporation buffer, and the output flow rate through the second outlet. It may be necessary to repeat this process with additional inlet and outlet pairs to continue to exchange the composition of the fluid from the first buffer to the electroporation buffer. It is possible that cells may also be lost to the outlet. It may be preferable to have the inlet on the floor side of the fluid channel and the outlet on the ceiling side with the outlet downstream of the inlet, so that the cells preferentially sediment by gravity toward the floor before encountering the outlet, such that this loss is minimized. In this case the distance between the inlets are set by the time for the cells to sediment a distance proportional to the channel height. It is possible that all of the inlets utilized for the buffer exchanged are connected to the same fluid control module. The same system described for exchanging buffer by inlet and outlet pairs could be used downstream of the region in which the cells are subject to an arbitrary voltage waveform to exchange the electroporation buffer for a recovery buffer. It is also conceivable that the molecules to be electroporated into the cells are contained in one of the secondary exchanged buffers.

### Example 8: Delivery of mRNA to Jurkat and Primary Pan T Cells

To demonstrate the capability of our exemplary device to deliver mRNA, we transfected Jurkat and primary T cells with mRNA encoding GFP. Primary T cells from healthy donors were transfected four days after activation with CD3/CD28 antibodies. Both cell types were resuspended at a concentration of 5×10⁶ cells/mL in low conductivity electroporation buffer containing GFP-encoding mRNA at either 20 μg/mL or 40 μg/mL, loaded into a syringe, and flowed into our electroporation flow cell as described in our methods. As cells transit under the electrode, the voltage temporal waveform was selected such that cells received three bipolar rectangular waveforms on average during their transit time through the electric field (f=100 μs, f=100 Hz). This value was inferred from the average linear velocity, the volumetric flow rate, and the dimension of the electrode along the direction of flow. As a control, cells were collected at zero applied voltage.

To measure delivery performance, cells were analyzed 24-h post-transfection using flow cytometry. For each voltage condition, cell count, viability, and GFP expression were directly measured through successive gating as described in the methods. Viability for each voltage condition was calculated as the number of viable cells (N_{viable}), measured using a viability dye (7-AAD), divided by the number of total cells in that voltage condition, measured 24-h post-transfection (Nₜₒₜₐₗ) (Eqn. 1).

$$Viability = \frac{{N}_{viable}}{{N}_{total}} \qquad Eqn . 1$$

For primary T cells, a viability ratio was calculated because the initial viability of each T cell donor ranged from 83-92%, independent of electroporation. Viability ratio was calculated as the viability (calculated in Eqn. 1) divided by the viability of the zero-voltage control cells (Viability_{zero-voltage control}) (Eqn. 2). The viability of zero-voltage control cells is measured 24-h post-transfection from cells that flowed through the device without an applied electric field.

$$Viability ratio = \frac{Viability}{{Viability}_{zero - voltage control}} \qquad Eqn . 2$$

GFP expression was defined as the number of viable and expressing cells (N_{expressing}) divided by the number of viable cells (Eqn. 3).

$$GRP expression = \frac{{N}_{expressing}}{{N}_{viable}} \qquad Eqn . 3$$

Since our measurement of viability does not account for cells that have been lost during electroporation (i.e. complete lysis), we also calculated relative yield as the total number of cells in each voltage condition (Nₜₒₜₐₗ) divided by the number of zero-voltage control cells (N_{zero-voltage control}) (Eqn. 4).

$$Relative yield = \frac{{N}_{total}}{{N}_{zero - voltage control}} \qquad Eqn . 4$$

Since relative yield is measured from cell counts 24-h post-transfection, this measurement is influenced by variation in cell seeding, variation in proliferation rate, and cell lysis or loss during electroporation.

We tested delivery of mRNA to Jurkat cells using either 20 or 40 μg/mL of mRNA encoding GFP and waveform voltage amplitude ranging from 3V to 11V. As the waveform voltage amplitude increased, we observed a threshold effect in which GFP expression was absent below 5V, then increased until reaching a plateau value of ˜97% at 11V. At all voltage amplitudes and mRNA concentrations tested, viability remained unchanged relative to zero-voltage controls while relative yield remained >90%. Notably, we observed 95%±1.8% GFP expression from primary T cells derived from four healthy donors, while the viability ratio and relative yield were 98%±1.4% and 92%±6.6%, respectively. As such, these data demonstrate the capability of our platform to deliver mRNA at high efficiency without impacting cell viability.

### Example 9: Controlling Fluids and Applied Voltages for Electroporation

Compared to the relevant systems for electroporation, such as those that use cylindrical pipette tips with electrodes on the inner surface of the pipette (see U.S. Pat. No. 9,523,071 B2 Pipette tip for electroporation device, which is incorporated by reference), with our essentially planar microfluidic chip-based flow system, cells experience a more uniform electric field. With the disclosed systems, we have the ability to change parameters during an electroporation run and we have the ability to scale the chip and process to treat the cells in the same manner with much larger sample volumes. This has the impact of facilitating both rapid tests to arrive at desirable conditions and then use the same underlying conditions for high-throughput processes such as cell manufacturing.

FIG. 1 shows a block diagram of a typical system we have used to test electroporation conditions. In this case we show a syringe pump that we have frequently used, but other types of pumps can also be used. For example, we have used pressure driven flow and the fluid flow rate can be measured and controlled by incorporation of a flow meter. FIG. 1 shows the system used to collect multiple samples that have been treated by application of different voltage waveforms. A multiwall plate is moved relative to the tubing output to sequentially collect different samples in different wells. The translation can be done manually, by a robotic device or other system for automated one- or two-dimensional translation.

### Example 10: Controlling Voltage Waveforms

We have used the disclosed (e.g., in Example 8) electrical test/analysis system for testing a range of voltage waveforms. We have capability to apply essentially arbitrary time dependent voltages to the chip and we can monitor electrical response of the system to monitor the process. In this large parameter space, we have focused on simple voltage shapes often involving an “opening” pulse and drive voltage, with long time average net zero current. Voltage conditions vary within a single run. The microfluidic chip design increases the number of parameters that can be tested in one run.

For example, we have used the disclosed systems to test the electroporation of Jurkat cells (Sigma) with Dasher mRNA coding for GFP (Aldevron). Cells were cultured in an incubator at 37° C. and 5% CO₂ and cultured in Dulbecco's modified Eagle's Minimum Essential Medium (DMEM, Sigma) supplemented with 10% fetal bovine serum (Sigma). The day before an electroporation experiment, the cells were seeded at a suspension density of 4×10⁵ cells/mL in culture media. On the day of electroporation, 16 million cells were withdrawn from the suspension and centrifuged at 500 g for 5 minutes. The cells were then washed two times in a proprietary, low conductivity electroporation buffer. After the second wash, cells were resuspended with 2 mL of electroporation buffer bringing the cell suspension density to 8×10⁶ cells/mL. mRNA was subsequently added to bring the concentration to 20 μg/mL.

The aqueous solution containing cells and plasmid DNA was loaded into a syringe which was then loaded into a syringe pump. Cells were flowed at 200 μL/minute and collected into individual wells of a 24-well plate for 15 seconds per waveform resulting in an initial seeding of 400,000 cells per experimental waveform. 24-well plates contained 0.5 mL of culture media resulting in an initial seeding density of 8×10⁵ cells/mL for each experimental waveform.

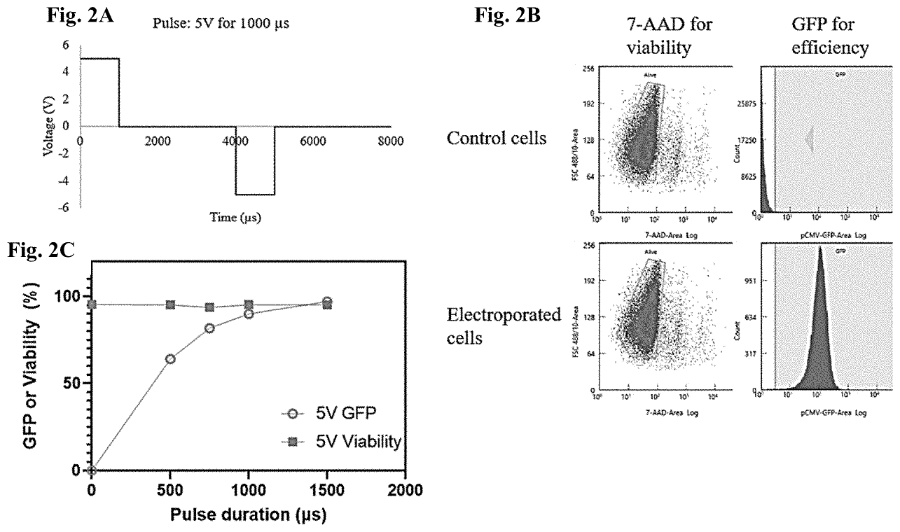

Experimental waveforms were designed to be bipolar (FIG. 2A) with a positive and negative polarity pulse of equal duration. The waveform had a period of 8 ms and repeated at 125 Hz. With the given flow rate and waveform period, cells received on average 1-2 pulses during their transit under the electrode. After electroporation, cells were immediately incubated at 37° C. and 5% CO₂. Cell viability was measured 24 hours after electroporation by incubation with the 7-AAD viability dye and measurement of positive staining cells via flow cytometry (FIG. 2B). Transfection efficiency was measured by GFP positive cells via flow cytometry (FIG. 2B). Control cells were subjected to similar conditions but received no electrical pulse.

Using a waveform that delivers a bipolar 5V pulse for 1500 μs, the disclosed electroporation platform was able to achieve 97% GFP expression with 95.2% viability compared to 0% GFP expression and 95.4% viability in the control cells that received no electrical pulse (FIG. 2C).

### Example 11: Variable Electroporation Parameters

The disclosed approach for flow electroporation has the novel capability to use multiple fluid inputs into the microfluidic chip. In earlier patent filings (see, e.g., US 2020/0399578; incorporated by reference) we have described the ability to independently control the composition of multiple flow streams. This enables laminar sheath flow such that the fluid streams do not mix significantly before the cells reach the region in the microfluidic device where the electric fields are applied. This capability enables multiple advantages such as keeping cells in buffer that preserves viability, separating cells from potentially cytotoxic electrodes or cytotoxic electrochemistry, and controlling the fluid conductivity. However, the advantages of controlling multiple streams do not necessarily require laminar or sheath flow. In some cases, it may be preferable to allow fluid inputs to mix or inter-diffuse before the fluids reach the region where the electrodes provide the porating electric fields. Lengthening the distance between where the fluids combine and the electrodes would allow more time and opportunity for interdiffusion of the fluids. Structures can also be introduced in the fluid channel to cause or enhance fluid mixing prior to the application of the electric field. Similarly, introducing fluid inputs after the application of the electric fields, where the different flows mix or inter-diffuse could be advantageous for enhancing the viability or health of the electroporated cells. It is also important to note that these advantages are not limited to thin planar microfluidic devices where the cells are in a single layer. There would be advantages for relatively wide channel with cells in various positions.

FIG. 3 shows a diagram of a system configuration in which the fluid composition is varied in addition to electrical parameters. The system has multiple independently controlled fluid sources. By varying the flow rates from the different fluid sources, different fluid compositions can be formed by combination near the electroporation chip input. The multiple flows, in one embodiment of the chip can form laminar layers of flow with different compositions in each layer to create sheath flow. In in addition to the chemical and electrical characteristics of each fluid, the amount of dissolved gas may be controlled to influence the formation or nucleation of gas bubbles or to control the amount of oxygen or other dissolved gas provided to the cells.

### Example 12: Electroporation Connection Features

In the previously described examples shown above there was a length of tubing used to connect the pump to the EP chip as well as tubing to convey the fluid output to the sample collection volume, typically a multi-well plate or tube(s). However, one or both tubing lengths may be eliminated by configuring the chip with integrated connection features.

The chip may be a single-use one (disposable device) that is kept sterile until use. Having this be a compact system that can be simply installed in a system would be desirable. This is similar to how easy and reliable it is to connect a USB drive to a computer. The encompassed chip connections could have a similar feel making simple electrical and fluidic connections. Eliminating the tubing also reduces unnecessary volume and surface area and reduces the time between electroporation and cell collection. The collection can be done, for example, into a well or container that contains cell growth medium, so the cells spend as little time as possible in other media such as those used for electroporation.

Some of the tested chips are made of plastic. Plastic can easily be formed in various shapes by well-established manufacturing processes such as injection molding. The chips can be configured with an integrated output device which we will here call a “nozzle” that has a lumen connected to the flow channel. It would be desirable to have the internal cross-sectional area of the nozzle lumen to be comparable to or slightly greater than the cross-sectional area of the fluidic channel. Our channel cross-sections are typically rectangular but the nozzle lumen could be circular or any other convenient shape. The external surface of the nozzle could be coated with hydrophobic material as desired to assist in drop formation. The nozzle could be on the planar face of the chip or on the end as shown in the diagrams in FIG. 4. In one embodiment, the nozzle is held over the collection volume and the fluid drops, by gravity, into the collection volume or well. It should be clear, however, that in other embodiments the nozzle could make contact with the collection volume and the fluid is driven by forces other than gravity into the collection volume. In an important application, the collection volume could be the input of an analytical system or further sample processing system or device.

### Example 13: Input and Output Connections

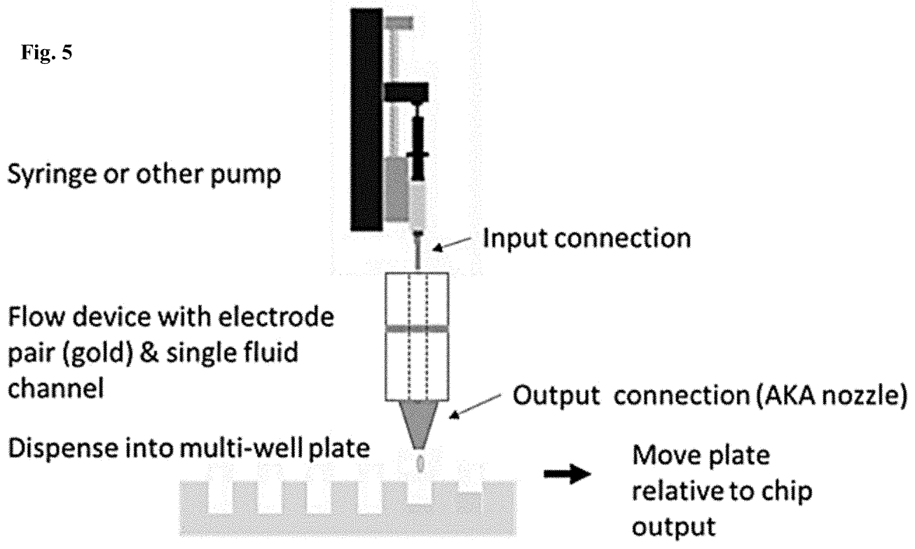

FIG. 5 shows a configuration of the system where the pump drives the fluid through a microfluidic electroporation chip with an integrated fluidic output structure or nozzle. This figure shows the nozzle on the chip edge, but it could also be on the planar face of the chip. Moving the collection volume relative to the nozzle facilitates dispensing samples, possibly treated by different voltage waveforms, into different wells such as those in a multi-well plate. The input connection can be made to an integrated chip structure and employ any standard fluid connection such as compression fittings or a Luer lock connection.

### Example 14: Flow Direction

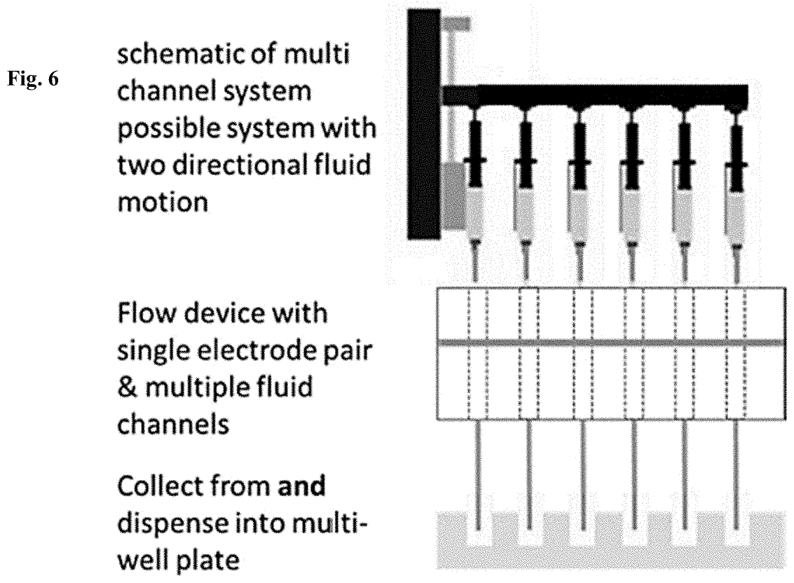

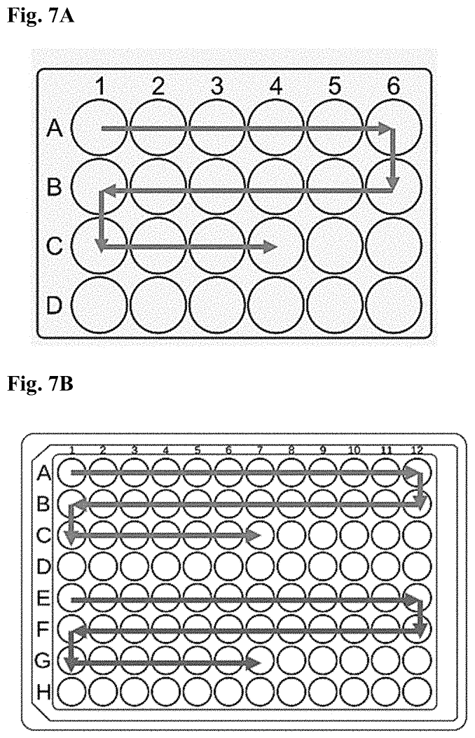

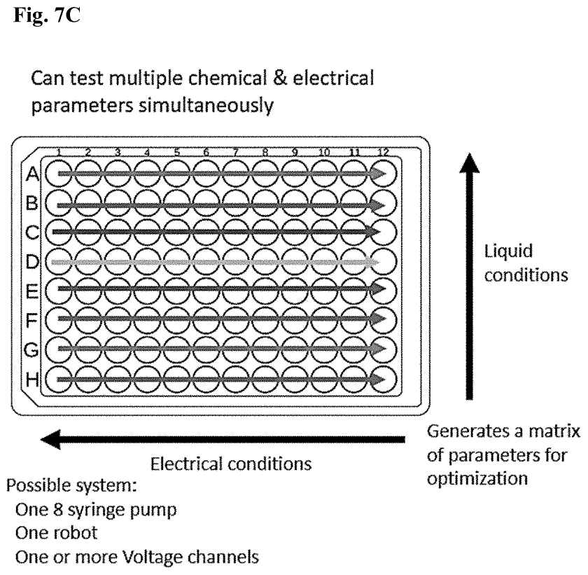

The other examples describe the flow chip as having a distinct input and output. However, flow can be driven in either direction by the pump(s). It may be desirable to input the fluid from multiple wells or tubes and electroporate the cells during flow in either direction. This allows a wide range of sample processing possibilities and use of robotic sampling automation processes. FIG. 6 depicts a system in which fluid can be driven in either direction by selection of the pump operation. This adds the capability to both collect and dispense fluid from a multi-well plate.

### Example 15: Automatable Electroporation Process

One example of readily automatable process steps would be:
1. Preload chip and syringe pump volumes with prescribed volume of cell growth medium.
2. Prepare a variety of cell samples with different electroporation reagents compositions in a multi-well plate using conventional automated processes.
3. Draw fluid from multiple wells in parallel through a multi-channel flow chip while applying voltage waveforms under the controlled flow. Agitation of plate may be used if desired.
4. Reverse flow and dispense the electroporated sample into a different receiving multi-well plate for culturing and/or analysis

The described approaches improve the ability to efficiently treat many samples in different ways, which is particularly valuable for biomedical research. The results of these tests and research may be a process that is valuable for cell therapies, and larger scale cell manufacturing using the derived conditions would be desired. With our system, as an example, multiple chips can be run in parallel to multiply the throughput. With these approaches we can scale processes as desired for a given application.

### Example 16: Rapid Optimization of Transfection Parameters Using Small Volumes of Reagents and Automation

To demonstrate our ability to rapidly optimize transfection of Jurkat cells, we tested electroporation of Jurkat cells (Sigma) with plasmid DNA encoding for green fluorescent protein (GFP) (Altogen Biosystems). Jurkat cells were purchased from Millipore Sigma, were cultured in an incubator at 37° C. and 5% CO₂, and cultured in RPMI 1640 media (R&D Systems) supplemented with 10% fetal bovine serum (R&D Systems).

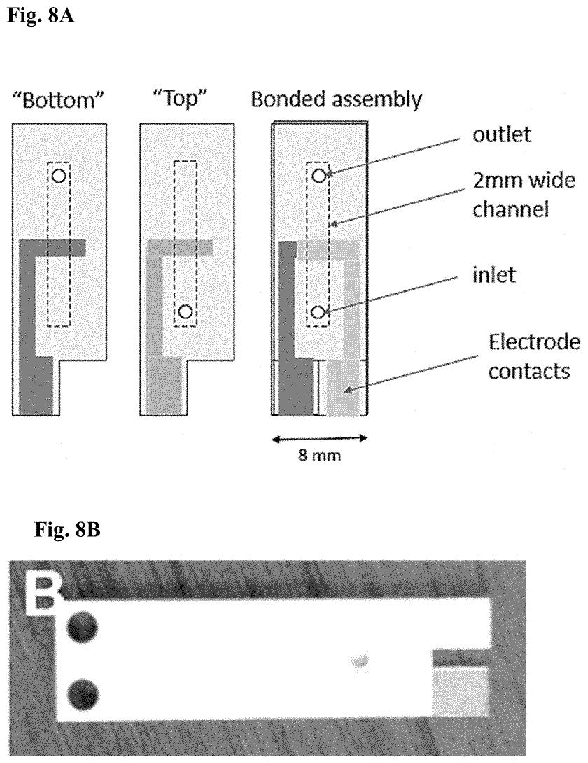

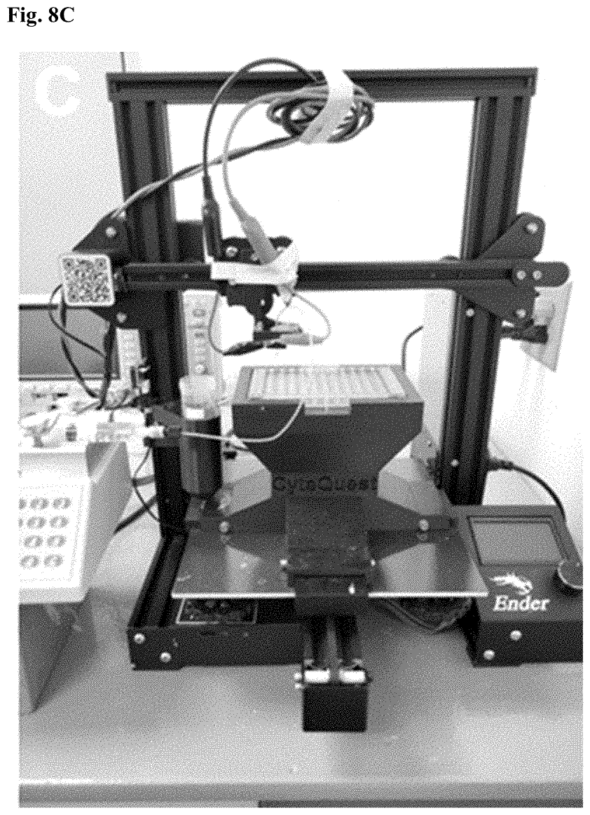

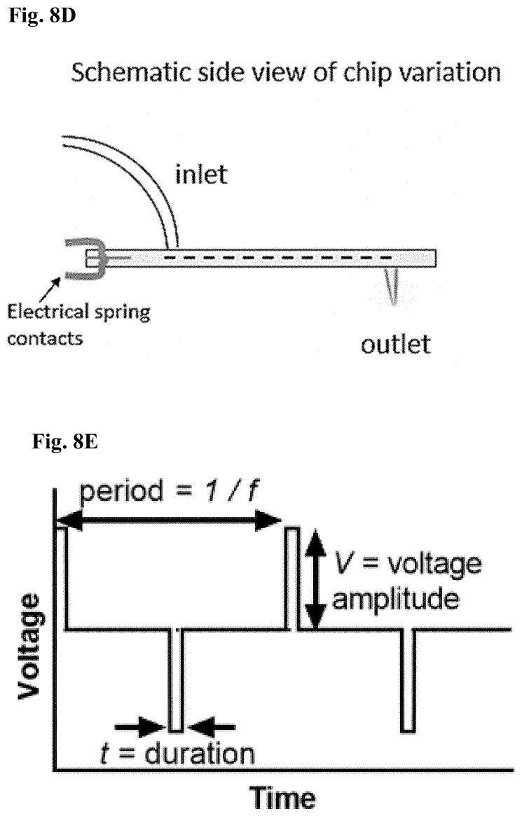

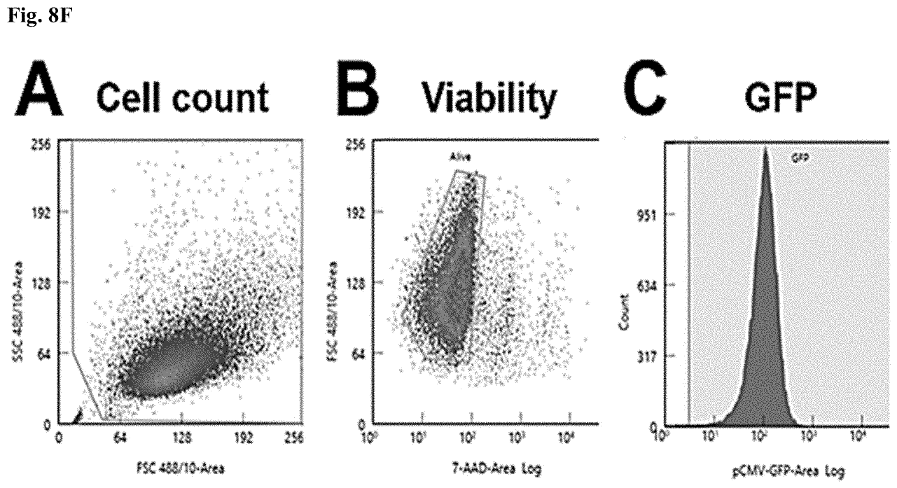

Jurkat cells were electroporated using an electroporation flow cell pictured in FIG. 8A. The flow cell was a sandwich assembly composed of two outer plastic layers and an inner spacer layer composed of an adhesive tape (Adhesives Research) (FIG. 8B). The inner adhesive layer was laser cut using a CO₂ laser to define the channel width as 2 mm while the height was defined as 100 μm by the inner adhesive layer's thickness. Inlet and outlet holes were cut in the outer plastic layers using laser cutting. The flow cell was mounted onto a manifold to which 0.02″ tubing was attached to both the inlet and outlet holes. The entire assembly was then mounted onto the arm of a robot capable of automated translation in the XZ axes (Creality) (FIG. 8C). A 96-well plate was mounted onto the stage of the same robot and was capable of automated translation in the Y axis. Together, the robot was capable of precise, automated motion in three axes.

Cells were harvested for electroporation, counted, and washed two times in a proprietary, low conductivity electroporation buffer. After the second wash, cells were resuspended in electroporation buffer at a density of 5×10⁶ cells/mL. Plasmid DNA was subsequently added to bring the concentration to 50 μg/mL. The aqueous solution containing cells and plasmid DNA was loaded into a syringe which was then loaded into the syringe pump (Microchem). The syringe pump was connected to the flow cell inlet via 0.02″ tubing. Cells were flowed at 500 μL/min and exited the channel via the outlet and 0.02″ outlet tubing.

Cells were collected into a 96-well plate. As a control, non-electroporated cells were collected first by keeping the electricity off. After collecting the control fraction, the electricity was switched on. Afterwards, electroporated fractions were collected over a roughly 5-minute experiment time. Cells were seeded for 2 s per well at a seeding density of 5×10⁵ cells/mL.

Time varying electrical waveforms were generated by a function generator (Siglent) and amplified by an amplifier (Accel Instruments). The time varying electrical waveform for electroporation was a bipolar square wave with a positive and negative polarity pulse of equal duration (FIG. 8E). The waveform was characterized by three parameters: waveform frequency (f), amplitude (V), and duration (t). For this optimization experiment, we tested f=33, 66, 100, or 133 Hz, V=20, 25, 30, or 35V, and t=75, 100, 125, 150, 175, or 200 μs. These 96 waveforms were split between two 96-well plates (48 waveforms per plate). Waveform switching was controlled by a MATLAB program (Mathworks) and coordinated with a program controlling the robot to ensure each well within the 96-well plate received one waveform. Precise seeding of each well within the 96-well plate was achieved using robotic translation of the outlet tubing in the XZ axes and robotic translation of the 96-well plate in the Y direction.

After electroporation, cells were immediately incubated at 37° C. and 5%. Cell count was measured by excluding debris through forward and side scattering signal via flow cytometry (FIG. 8F, panel A). Cell viability was measured by incubation with the 7-AAD viability dye and measurement of positive staining cells via flow cytometry (FIG. 8F, panel B). Transfection efficiency was measured by live-gated GFP positive cells via flow cytometry (FIG. 8F, panel C). Thresholds for GFP expression was determined using live-gated control cells that did not experience an electrical waveform.

As shown in Table 2, the time varying electrical waveforms spanned a wide range of values for transfection efficiency and viability. This enabled rapid selection of the time varying electrical waveform to meet desired values for transfection efficiency and cellular viability.

TABLE 2
Results from optimization of time-varying electrical waveform

| 33 Hz | 75 | 100 | 125 | 150 | 175 | 200 | 33 Hz | 75 | 100 | 125 | 150 | 175 | 200 |
|---|---|---|---|---|---|---|---|---|---|---|---|---|---|
| GFP | us | us | us | us | us | us | Viability | us | us | us | us | us | us |
| 20 V | 14% | 22% | 29% | 34% | 39% | 44% | 20 V | 97% | 95% | 96% | 96% | 95% | 94% |
| 25 V | 26% | 36% | 44% | 48% | 55% | 60% | 25 V | 96% | 96% | 96% | 95% | 94% | 93% |
| 30 V | 38% | 48% | 56% | 61% | 66% | 69% | 30 V | 97% | 96% | 95% | 94% | 94% | 91% |
| 35 V | 45% | 55% | 63% | 69% | 73% | 75% | 35 V | 96% | 95% | 94% | 93% | 92% | 89% |
| 66 Hz | 75 | 100 | 125 | 150 | 175 | 200 | 66 Hz | 75 | 100 | 125 | 150 | 175 | 200 |
| GFP | us | us | us | us | us | us | Viability | us | us | us | us | us | us |
| 20 V | 22% | 30% | 38% | 44% | 49% | 52% | 20 V | 94% | 94% | 91% | 92% | 90% | 86% |
| 25 V | 34% | 45% | 52% | 59% | 65% | 70% | 25 V | 93% | 92% | 90% | 89% | 82% | 77% |
| 30 V | 44% | 55% | 63% | 70% | 74% | 78% | 30 V | 92% | 90% | 87% | 80% | 70% | 68% |
| 35 V | 51% | 63% | 68% | 78% | 80% | 82% | 35 V | 91% | 86% | 78% | 70% | 58% | 53% |
| 100 Hz | 75 | 100 | 125 | 150 | 175 | 200 | 100 Hz | 75 | 100 | 125 | 150 | 175 | 200 |
| GFP | us | us | us | us | us | us | Viability | us | us | us | us | us | us |
| 20 V | 48% | 57% | 64% | 68% | 71% | 74% | 20 V | 96% | 91% | 88% | 85% | 83% | 72% |
| 25 V | 61% | 68% | 74% | 78% | 82% | 83% | 25 V | 92% | 83% | 77% | 75% | 68% | 55% |
| 30 V | 68% | 76% | 80% | 83% | 86% | 87% | 30 V | 88% | 72% | 69% | 64% | 56% | 42% |
| 35 V | 74% | 81% | 79% | 87% | 89% | 90% | 35 V | 83% | 70% | 55% | 53% | 43% | 36% |
| 133 Hz | 75 | 100 | 125 | 150 | 175 | 200 | 133 Hz | 75 | 100 | 125 | 150 | 175 | 200 |
| GFP | us | us | us | us | us | us | Viability | us | us | us | us | us | us |
| 20 V | 43% | 52% | 57% | 62% | 66% | 72% | 20 V | 91% | 90% | 83% | 79% | 76% | 74% |
| 25 V | 53% | 63% | 71% | 75% | 78% | 82% | 25 V | 87% | 83% | 81% | 71% | 63% | 64% |
| 30 V | 64% | 72% | 78% | 82% | 83% | 86% | 30 V | 85% | 79% | 72% | 53% | 48% | 51% |
| 35 V | 71% | 79% | 83% | 87% | 87% | 87% | 35 V | 80% | 70% | 55% | 40% | 37% | 33% |

### Example 17: Rapid, Multiplexed Optimization of Transfection Parameters

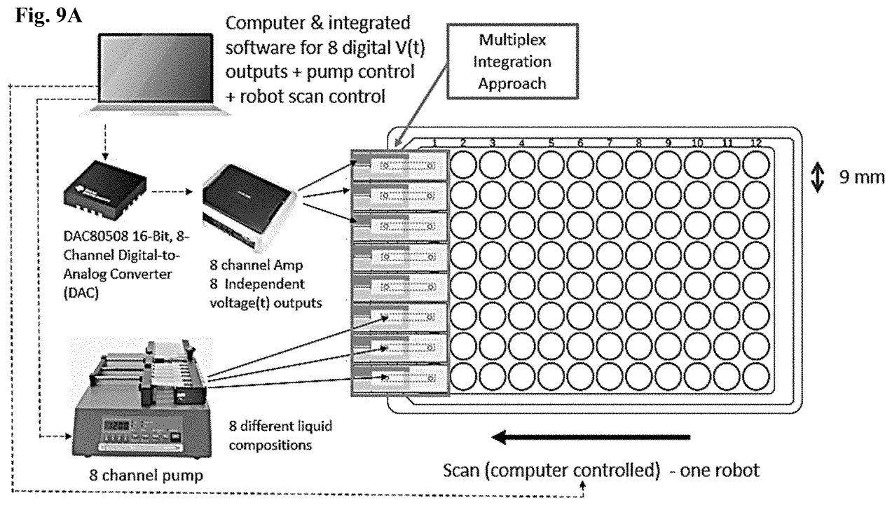

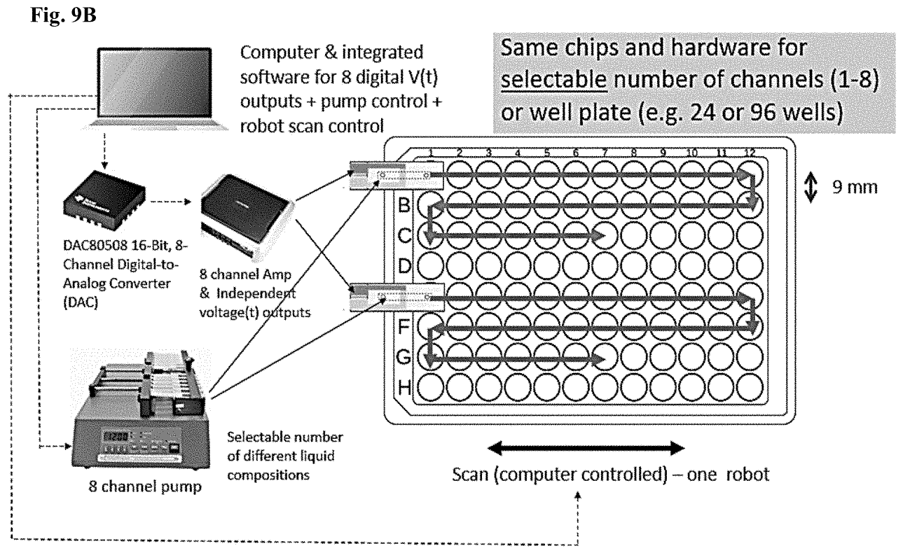

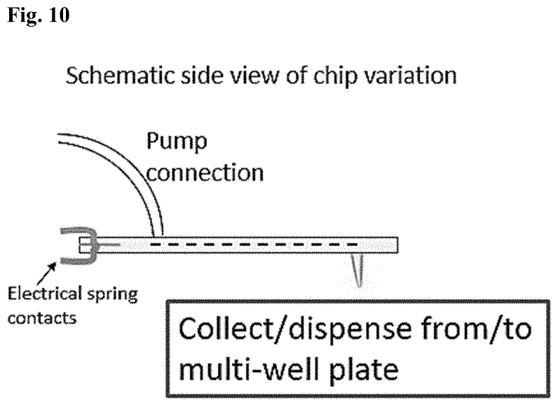

To demonstrate our ability to rapidly optimize transfection, we describe how we can vary multiple parameters within a single experiment including: cell type(s), cell concentration in the electroporation buffer, cargo type(s), cargo concentration in the electroporation buffer, and electroporation composition. Up to 8 unique and separate combinations of the above-described parameter set can be tested simultaneously in a single experiment using the system shown in FIG. 9A.

Each of the 8 unique mixtures are created by resuspending cells within electroporation buffer and adding the desired cargo. These are loaded into separate syringes, which are then loaded into an 8-channel syringe pump. The syringe pump next pumps these mixtures through tubing attached to the inlets of 8 separate electroporation chips (FIG. 9A). Cells transit under the electrode, receive a time varying electrical waveform, then exit the outlet through a hydrophobic nozzle that encourages drop formation.

Electroporation chips are designed with outer dimensions of 8 mm (width) by 28 mm (length). The 8 mm width ensures that an array of flow cells is able to simultaneously seed 8 wells of a 96-well plate as shown in FIG. 9A.

Time varying electrical waveforms are generated by a computer and integrated software capable of 8 digital outputs. They input into a DAC80508 16-bit 8-channel digital-to-analog converter which then input into an 8-channel amplifier that produce 8 independent time varying electrical waveforms. The computer and integrated software also control the 8-channel syringe pump and robot, which enables each well to receive a mostly pure population of cells exposed to a single time varying electrical waveform. Using this program, each of the 8 unique cell/cargo mixtures can receive up to 12 different time varying electrical waveforms for a total of 96 different conditions. The 12-time varying electrical waveforms do not need to be the same for each of the 8 unique cell/cargo mixtures.

As drops exit the nozzle, they are deposited into a 96-well plate containing RPMI 1640 media warmed to 37 degrees Celsius. The 96-well plate is mounted within a robotic stage capable of translation in the XYZ-axes. As the syringe pump drives cells/cargo through each of the 8 chips, the robotic stage, controlled by the computer program, translates the 96-well in the X-axis relative to the flow cell output to collect drops into each of the 12 rows of the 96-well plate. For instance, row 1 has 8 wells and receives cells from each of the 8 different cell/cargo mixtures exposed to time varying waveforms 1-12. Row 2 receives cells from each of the 8 different cell/cargo mixtures exposed to time varying waveforms 13-24. This continues for rows 3 through 12.

After all wells of the 96-well plate have been seeded, the 96-well plate is moved to an incubator that maintains the cells at 37° C. and 5% CO₂. Cells are analyzed for expression of cargo and viability at an appropriate interval (i.e., 24 hours post-transfection).

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present invention described herein. Such equivalents are intended to be encompassed by the following claims.

1. A flow device for electroporation, comprising
at least one planar flow channel;
at least one pair of electrodes;
at least one first port; and
at least one second port.

2. The flow device of claim 1, wherein the at least one second port is located:
(a) on the bottom of the flow device (e.g., FIG. 4A) such that the flow from the second port is perpendicular to the flow within the channel; or
(b) on the side of the flow device (e.g., FIG. 4B) such that the flow from the second port is in the same direction as the flow within the channel.

3. The flow device of claim 1, wherein
(a) the width of the device is at least about 1, 2, 3, 4, 5, 6, 7, or 8 mm but no more than about 9, 10, 11, 12, 13, 14, 15, 16, 17, 18, 19, or 20 mm, optionally wherein the width of the device is about 8 mm;
(b) the width of the channel is at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, or 7 mm, optionally wherein the width of the channel is at least about 2 mm but no more than about 7 mm, optionally wherein the width of the channel is about 2 mm;
(c) the at least one pair of electrodes extends to at least one edge of the flow device, optionally distal to the at least one second port; and/or
(d) the at least one first port is located on the bottom of the flow device, on top of the flow device, on the side of the flow device, or any combination thereof.

4.-8. (canceled)

9. A microfluidic flow-based electroporation system, comprising
at least one flow device of claim 1;
at least one electrical control module that connects to the pair of electrodes;
at least one fluid delivery system that links to the flow device at the at least one first port; and
a multi-well module that links to the flow device at the at least one second port.

10. The system of claim 9, wherein
(a) the system comprises at least 1, 2, 3, 4, 5, 6, 7, 8, 9, 10, 11, 12, 13, 14, 15, or 16 flow devices, optionally 8 flow devices;
(b) the flow device is positioned horizontally or vertically with respect to the multi-well module;
(c) the multi-well module comprises a 6-, 24-, 48-, 96-, or 384-well plate; and/or
(d) the at least one pair of electrodes is connected to the at least one electrical control module from the top, bottom, or the side of the flow device.

11.-14. (canceled)

15. The system of claim 9, wherein the fluid delivery module
(a) allows moving fluid from a first port toward a second port, from a second port toward a first port, or from either port toward the other port;
(b) comprises a syringe or a pump; and/or
(c) connects to a flow device at a first port via a tubing; and/or a multi-well module connects to a second port via a tubing.

16.-17. (canceled)

18. The system of claim 9, further comprising
(a) a robotic module that moves the multi-well module relative to the flow device;
(b) a robotic module that moves the flow device relative to the multi-well module; or
(c) a robotic module selected from any one of the robotic modules listed in Table 1.

19.-20. (canceled)

21. The system of claim 9, wherein the at least one electrical control module
(a) provides a voltage that has a bipolar square wave, a dual voltage waveform, periodic waveform, or a periodic arbitrary time-varying voltage, optionally a voltage that has a period waveform;
(b) is connected to the at least one pair of electrodes independently from any other pair of electrodes; and/or
(b) allows forming an electric field as a function of time and/or position within the fluid channel.

22. (canceled)

23. The system of claim 21, wherein:
(a) the periodic waveform is a sinusoidal function of time, wherein the sinusoidal function has an absolute amplitude from zero that is at most 200 Volts, a frequency that is at least 10 Hz and at most 100 kHz, and a phase that is at least 0 and at most 2n;
(b) the periodic waveform has a first frequency and a second frequency different from the first frequency;
(c) the periodic waveform is a Fourier series; and/or
(d) the periodic waveform is a square waveform or a rectangular waveform having a voltage amplitude of at least 0.1 V and at most 100 V, and a frequency of at least 100 Hz and at most 1 THz.

24. The system of claim 23, wherein:
(a) the square waveform or a rectangular waveform is bipolar; and/or
(b) the square waveform or a rectangular waveform further comprises a direct current component of at most ±10 V.

25. (canceled)

26. The system of claim 9, wherein the flow device comprises at least two first ports and at least two fluid delivery systems, wherein each fluid delivery system is connected to a different first port, optionally wherein the fluid delivery system allows modulating the flow rate and chemical composition in one of the at least two streams as a predetermined function of time, position, or both time and position independently from any other fluid stream within the channel.

27. (canceled)

28. The system of claim 9, further comprising:
(a) a flow sensor;
(b) a flow-rate control module;
(c) a temperature control module;
(d) a fluid interface that couples the fluid delivery system to the flow device;
(e) a cell processing module;
(f) an electrical or optical monitoring module coupled to the flow device; or
(g) any combination of two or more of (a)-(f).

29. The system of claim 28, wherein the cell processing module
(a) is upstream from the flow device;
(b) allows cell sorting, selection, labeling, analysis, or a combination thereof;
(c) comprises a fluorescence-activated cell sorting component;
(d) comprises a magnetic field source that allows magnetic bead separation; and/or
(e) is built in the device (e.g., FIG. 11) or built in another microfluidic device (e.g., FIG. 12).

30.-33. (canceled)

34. The system of claim 28, further comprising an apheresis bag upstream of the cell processing module.

35. A method of electroporating a cell, comprising flowing a cell through the flow device or the system of claim 9, and applying voltage to the electrodes.

36. A method of identifying at least one suitable electroporation parameter, the method comprising
(a) electroporating cells according to the method of claim 35, under at least one parameter;
(b) electroporating cells under at least one additional parameter;
(c) comparing the transfection efficiency, cell viability, or both of the cells in (a) and (b); and
(d) identifying an electroporation parameter that yields a higher transfection efficiency, cell viability, or both as a suitable electroporation parameter.

37. The method of claim 36, wherein
(a) the at least one electroporation parameter comprises cell type, cell concentration, heterologous object type, heterologous object concentration, fluid flow rate, fluid volume, buffer type, time-dependent voltage waveform, collection time, or a combination of two or more thereof;
(b) the at least one electroporation parameter comprises cell type, cell concentration, heterologous object type, heterologous object concentration, fluid flow rate, fluid volume, buffer type, time-dependent voltage waveform, and collection time;
(c) the cells are electroporated using a system comprising at least 2 flow devices, optionally 8 flow devices;
(d) the cells are electroporated using a system comprising a multi-well module, optionally wherein the multi-well module comprises a 98-well plate; or
(e) the cells are electroporated using a system comprising a robotic module.

38. (canceled)

39. The method of claim 37, wherein the cell type, cell concentration, heterologous object type, heterologous object concentration, fluid flow rate, fluid volume, buffer type, or a combination of two or more thereof is kept constant.

40.-42. (canceled)

43. The method of claim 35, wherein
(a) the method is for manufacturing cells for cellular therapies, optionally wherein the cellular therapies comprises a CAR therapy;
(b) the method electroporates cells with a heterologous object; and/or
(c) the cell is a mammalian cell, a human cell, a lymphocyte, or a T cell, or a primary T cell.

44.-47. (canceled)

48. The method of claim 43, wherein
(a) the heterologous object comprises a nucleic acid, an mRNA, or a CRISPR/Cas9 RNP; and/or
(b) the method modifies a genome of the cell.

49.-51. (canceled)
